SEQUENCE LISTING (10 sequences)

SEQ ID NO: 1 - protein, 5 aa, Artificial Sequence
  CDR1-H of huDS6
SYNMH

SEQ ID NO: 2 - protein, 17 aa, Artificial Sequence
  CDR2-H of huDS6
YIYPGNGATNYNQKFQG

SEQ ID NO: 3 - protein, 8 aa, Artificial Sequence
  CDR3-H of huDS6
GDSVPFAY

SEQ ID NO: 4 - protein, 10 aa, Artificial Sequence
  CDR1-L of huDS6
SAHSSVSFMH

SEQ ID NO: 5 - protein, 7 aa, Artificial Sequence
  CDR2-L of huDS6
STSSLAS

SEQ ID NO: 6 - protein, 9 aa, Artificial Sequence
  CDR3-L of huDS6
QQRSSFPLT

SEQ ID NO: 7 - protein, 117 aa, Artificial Sequence
  Heavy chain variable region of huDS6
QAQLVQSGAEVVKPGASVKMSCKASGYTFTSYNMHWVKQTPGQGLEWIGYIYPGNGATNYNQKFQGKATLTADPSSSTAYMQISSLTSEDSAVYFCARGDSVPFAYWGQGTLVTVSA

SEQ ID NO: 8 - protein, 107 aa, Artificial Sequence
  Light chain variable region of huDS6
EIVLTQSPATMSASPGERVTITCSAHSSVSFMHWFQQKPGTSPKLWIYSTSSLASGVPARFGGSGSGTSYSLTISSMEAEDAATYYCQQRSSFPLTFGAGTKLELKR

SEQ ID NO: 9 - protein, 447 aa, Artificial Sequence
  Heavy chain of huDS6
QAQLVQSGAEVVKPGASVKMSCKASGYTFTSYNMHWVKQTPGQGLEWIGYIYPGNGATNYNQKFQGKATLTADPSSSTAYMQISSLTSEDSAVYFCARGDSVPFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 213 aa, Artificial Sequence
  Light chain of huDS6
EIVLTQSPATMSASPGERVTITCSAHSSVSFMHWFQQKPGTSPKLWIYSTSSLASGVPARFGGSGSGTSYSLTISSMEAEDAATYYCQQRSSFPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2016/0347856 A1
(43) Pub. Date: Dec. 1, 2016

(54) Use of Anti-MUC1 Maytansinoid Immunoconjugate Antibody for the Treatment of Solid Tumors

(71) Applicant: SANOFI, Paris (FR)

(72) Inventors: Sylvie Assadourian, Paris (FR); Dominique Mignard, Paris (FR)

(21) Appl. No.: 14/909,671

(22) PCT Filed: Jul. 30, 2014
(86) PCT No.: PCT/EP2014/066345

§ 371 Date: Feb. 2, 2016

(30) Foreign Application Priority Data
Aug. 2, 2013 (EP) 13306119.2

(51) Int. Cl. C07K 16/30; A61K 47/48

(52) U.S. Cl. CPC C07K 16/3092; A61K 47/48569; A61K 47/48384; C07K 2317/565; C07K 2317/56

(57) ABSTRACT

The present invention concerns a conjugate comprising (i) a cell binding agent which binds to the human mucin-1 (MUC1) glycoprotein, linked to (ii) at least one cytotoxic agent, for use to treat cancer, wherein said conjugate is administered at a dose of at least 120 mg/m².

### FIELD OF THE INVENTION

The present invention concerns a conjugate comprising (i) a cell binding agent which binds to the human mucin-1 (MUC1) glycoprotein, linked to (ii) at least one cytotoxic agent, for use to treat cancer, wherein said conjugate is administered at a dose of at least 120 mg/m².

### BACKGROUND OF THE INVENTION

There have been numerous attempts to develop anti-cancer therapeutic agents that specifically destroy target cancer cells without harming surrounding, non-cancerous cells and tissues. Such therapeutic agents have the potential to vastly improve the treatment of cancer in human patients.

One promising approach has been to link cell binding agents, such as monoclonal antibodies, with cytotoxic drugs. Depending on the selection of the cell binding agent, these cytotoxic conjugates can be designed to recognize and bind only specific types of cancerous cells, based on the expression profile of molecules expressed on the surface of such cells.

The international patent application WO 02/16401 described a murine monoclonal antibody DS6 which reacts with an antigen, CA6 that is expressed by human serous ovarian carcinomas. This murine monoclonal antibody DS6 can therefore target cancerous cells.

The CA6 antigen was more specifically characterized in the U.S. Pat. No. 7,834,155, as a sialoglycotope on the MUC1 mucin receptor expressed by cancerous cells. This patent also provided antibodies, in particular humanized antibodies such as the humanized hDS6 antibody, capable of recognizing this CA6 sialoglycotope of the MUC1 mucin receptor.

Cytotoxic drugs such as methotrexate, daunorubicin, doxorubicin, vincristine, vinblastine, melphalan, mitomycin C, and chlorambucil have been used in cytotoxic conjugates, linked to a variety of murine monoclonal antibodies. In some cases, the drug molecules were linked to the antibody molecules through an intermediary carrier molecule such as serum albumin.

The development of cytotoxic conjugates that specifically recognize particular types of cancerous cells will be important in the continuing improvement of methods used to treat patients with cancer.

To that end, the present invention is directed to the development of conjugates comprising cell binding agents, such as antibodies, and cytotoxic agents that specifically target the molecules/receptors expressed on the surface of cancerous cells.

More specifically, the present invention is directed to conjugates comprising antibodies, preferably humanized antibodies, that recognize the CA6 sialoglycotope of the Mud mucin receptor expressed by cancerous cells and that may be used to inhibit the growth of a cell expressing the CA6 glycotope in the context of a cytotoxic agent. One of these conjugates is SAR566658.

SAR566658 is an immunoconjugate consisting of a humanized monoclonal antibody against the tumor-associated sialoglycotope CA6 (huDS6) conjugated to the cytotoxic maytansinoid DM4.

More particularly, the present invention provides cytotoxic conjugates that recognize the CA6 sialoglycotope of the Mud mucin receptor, for which it was necessary to determine the suitable dose of administration and regimen in order to obtain a well-tolerated anti-cancer treatment which enables treating patients suffering from cancer, in particular patients suffering from CA6-positive cancers, in particular breast cancer or ovarian cancer.

### SUMMARY OF THE INVENTION

The present invention thus concerns a conjugate comprising (i) a cell binding agent which binds to the human mucin-1 (MUC1) glycoprotein, linked to (ii) at least one cytotoxic agent, for use to treat cancer, wherein said conjugate is administered at a dose of at least 120 mg/m².

The present invention also concerns a conjugate comprising (i) a cell binding agent which binds to the human mucin-1 (MUC1) glycoprotein, linked to (ii) at least one cytotoxic agent, for use to treat a cancer selected from the group consisting of breast cancer and ovarian cancer.

In some embodiments of the invention, the cell binding agent is a humanized anti-CA6 antibody and the cytotoxic agent is a maytansinoid.

In further embodiments, the cell binding agent is the humanized anti-CA6 antibody huDS6 comprising a heavy chain of sequence SEQ ID NO: 9 and a light chain of sequence SEQ ID NO: 10 and the cytotoxic agent is a maytansine compound such as DM1 or DM4.

In a particular embodiment, the conjugate used in the context of the invention is the compound SAR566658 of the following formula (XXI)

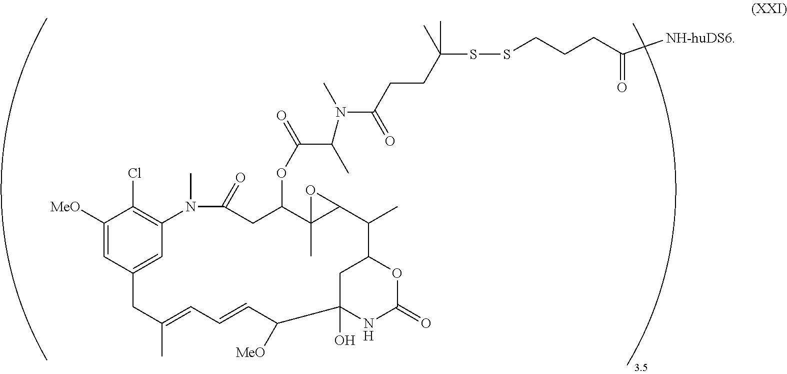

The present invention also concerns an article of manufacture comprising:

a) a packaging material;

b) a conjugate comprising (i) a cell binding agent which binds to the human mucin-1 (MUC1) glycoprotein, linked to (ii) at least one cytotoxic agent; more particularly the compound SAR566658 of formula (XXI), and

c) a label or package insert contained within said packaging material indicating that said conjugate is administered at a dose of at least 120 mg/m².

The present invention also concerns an article of manufacture comprising:

a) a packaging material;

b) a conjugate comprising (i) a cell binding agent which binds to the human mucin-1 (MUC1) glycoprotein, linked to (ii) at least one cytotoxic agent; more particularly the compound SAR566658 of formula (XXI), and

c) a label or package insert contained within said packaging material indicating that said conjugate is administered for treating a cancer selected from the group consisting of breast cancer and ovarian cancer.

### DETAILED DESCRIPTION OF THE INVENTION

### Definitions

In the context of the invention, the term “MUC1 glycoprotein” refers to a mucin encoded by the MUC1 gene in humans. MUC1 is a glycoprotein with extensive O-linked glycosylation of its extracellular domain. MUC1 has a core protein mass of 120-225 kDa which increases to 250-500 kDa with glycosylation. It extends 200-500 nm beyond the surface of the cell. The protein is anchored to the apical surface of many epithelia by a transmembrane domain. Beyond the transmembrane domain is a SEA domain that contains a cleavage site for release of the large extracellular domain. The extracellular domain includes a 20 amino acid variable number tandem repeat (VNTR) domain, with the number of repeats varying from 20 to 120 in different individuals. These repeats are rich in serine, threonine and proline residues which permits heavy O-glycosylation.

In the context of the invention, the term “CA6 glycotope” or “CA6 sialoglycotope” refers to a tumor-associated antigen present on the extracellular domain of the MUC1 glycoprotein, which was identified by Kearse et al. (2000) Int. J. Cancer. 88:866-872, as bearing a carbohydrate epitope that is sialic acid-dependent.

As used herein, a sequence “at least 85% identical to a reference sequence” is a sequence having, on its entire length, 85%, or more, in particular 90%, 91%, 92%, 93%, 94%, 95%, 96%, 97%, 98%, 99%, 99.5%, 99.6%, 99.7%, 99.8%, 99.9% A or 100% sequence identity with the entire length of the reference sequence.

A percentage of “sequence identity” may be determined by comparing the two sequences, optimally aligned over a comparison window, wherein the portion of the polypeptide sequence in the comparison window may comprise additions or deletions (i.e. gaps) as compared to the reference sequence (which does not comprise additions or deletions) for optimal alignment of the two sequences. The percentage is calculated by determining the number of positions at which the identical amino acid residue occurs in both sequences to yield the number of matched positions, dividing the number of matched positions by the total number of positions in the window of comparison and multiplying the result by 100 to yield the percentage of sequence identity. Optimal alignment of sequences for comparison is conducted by global pairwise alignment, e.g. using the algorithm of Needleman and Wunsch (1970) J. Mol. Biol. 48: 443. The percentage of sequence identity can be readily determined for instance using the program Needle, with the BLOSUM62 matrix, and the following parameters gap-open=10, gap-extend=0.5.

In the context of the invention, a “conservative amino acid substitution” is one in which an amino acid residue is substituted by another amino acid residue having a side chain group with similar chemical properties (e.g., charge or hydrophobicity). In general, a conservative amino acid substitution will not substantially change the functional properties of a protein. Examples of groups of amino acids that have side chains with similar chemical properties include 1) aliphatic side chains: glycine, alanine, valine, leucine, and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartic acid and glutamic acid; and 7) sulfur-containing side chains: cysteine and methionine. Conservative amino acids substitution groups are: valine-leucine-isoleucine, phenylalanine-tyrosine-tryptophane, lysine-arginine, alanine-valine, glutamate-aspartate, and asparagine-glutamine.

As used herein, the term “subject” denotes a mammal, such as a rodent, a feline, a canine, and a primate. In particular a subject according to the invention is a human.

As used herein, “conjugate”, “immunoconjugate”, “antibody-drug conjugate” or “ADC” have the same meaning and are interchangeable.

Throughout the instant application, the term “comprising” is to be interpreted as encompassing all specifically mentioned features as well optional, additional, unspecified ones. As used herein, the use of the term “comprising” also discloses the embodiment wherein no features other than the specifically mentioned features are present (i.e. “consisting of”).

### Cell Binding Agent

As used herein, the term “cell binding agent” refers to an agent that specifically recognizes and binds the human mucin-1 (MUC1) glycoprotein on the cell surface. In a particular embodiment, the cell binding agent binds, more particularly specifically binds, the extracellular domain of the MUC1 glycoprotein as defined in the section “Definition” hereabove. In another embodiment, the cell binding agent recognizes and binds the CA6 glycotope on the MUC1 glycoprotein as defined in the section “Definition” hereabove.

In one embodiment, the cell binding agent specifically recognizes the human MUC1 glycoprotein, in particular the extracellular domain of the MUC1 glycoprotein, more particularly the CA6 glycotope on the MUC1 glycoprotein, such that it allows the conjugates to act in a targeted fashion with little side-effects resulting from non-specific binding.

In another embodiment, the cell binding agent of the present invention also specifically recognizes the human MUC1 glycoprotein, in particular the extracellular domain of the MUC1 glycoprotein, more particularly the CA6 glycotope on the MUC1 glycoprotein, so that the conjugate will be in contact with the target cell for a sufficient period of time to allow the cytotoxic agent portion of the conjugate to act on the cell, and/or to allow the conjugates sufficient time in which to be internalized by the cell.

The effectiveness of the conjugates of the present invention as therapeutic agents depends on the careful selection of an appropriate cell binding agent which binds to the human mucin-1 (MUC1) glycoprotein, in particular to the extracellular domain of the MUC1 glycoprotein, more particularly to the CA6 glycotope on the MUC1 glycoprotein. Cell binding agents may be of any kind presently known, or that become known and includes peptides and non-peptides, as long as they bind to the human MUC1 glycoprotein, in particular to the extracellular domain of the MUC1 glycoprotein, more particularly to the CA6 glycotope on the MUC1 glycoprotein. Generally, these can be antibodies (especially monoclonal antibodies), lymphokines, hormones, growth factors, vitamins, nutrient-transport molecules (such as transferrin), or any other cell binding molecule substance.

More specific examples of cell binding agents that can be used include:

polyclonal antibodies;

monoclonal antibodies;

epitope-binding fragments of antibodies such as Fab, Fab′, F(ab′)₂ or Fv.

Selection of the appropriate cell binding agent is a matter of choice that depends upon the particular cell population that is to be targeted, but in general, antibodies or epitope-binding fragments thereof are preferred if an appropriate one is available or can be prepared, more preferably a monoclonal antibody.

An “antibody” may be a natural or conventional antibody in which two heavy chains are linked to each other by disulfide bonds and each heavy chain is linked to a light chain by a disulfide bond. There are two types of light chain, lambda (2) and kappa (κ). There are five main heavy chain classes (or isotypes) which determine the functional activity of an antibody molecule: IgM, IgD, IgG, IgA and IgE. Each chain contains distinct sequence domains. The light chain includes two domains or regions, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) influence the overall domain structure and hence the combining site.

“Complementarity Determining Regions” or “CDRs” refer to amino acid sequences which together define the binding affinity and specificity of the natural Fv region of a native immunoglobulin binding site. The light and heavy chains of an immunoglobulin each have three CDRs, designated CDR1-L, CDR2-L, CDR3-L and CDR1-H, CDR2-H, CDR3-H, respectively. A conventional antibody antigen-binding site, therefore, includes six CDRs, comprising the CDR set from each of a heavy and a light chain V region.

“Framework Regions” (FRs) refer to amino acid sequences interposed between CDRs, i.e. to those portions of immunoglobulin light and heavy chain variable regions that are relatively conserved among different immunoglobulins in a single species. The light and heavy chains of an immunoglobulin each have four FRs, designated FR1-L, FR2-L, FR3-L, FR4-L, and FR1-H, FR2-H, FR3-H, FR4-H, respectively.

As used herein, a “human framework region” is a framework region that is substantially identical (about 85%, or more, in particular 90%, 95%, 97%, 99% or 100%) to the framework region of a naturally occurring human antibody.

In the context of the invention, CDR/FR definition in an immunoglobulin light or heavy chain is to be determined based on IMGT definition (Lefranc et al. (2003) Dev Comp Immunol. 27(1):55-77; www.imgt.org).

As used herein, the term “antibody” denotes conventional antibodies and fragments thereof, as well as single domain antibodies and fragments thereof, in particular variable heavy chain of single domain antibodies, and chimeric, humanised, bispecific or multispecific antibodies.

As used herein, antibody or immunoglobulin also includes “single domain antibodies” which have been more recently described and which are antibodies whose complementary determining regions are part of a single domain polypeptide. Examples of single domain antibodies include heavy chain antibodies, antibodies naturally devoid of light chains, single domain antibodies derived from conventional four-chain antibodies, engineered single domain antibodies. Single domain antibodies may be derived from any species including, but not limited to mouse, human, camel, llama, goat, rabbit and bovine.

Single domain antibodies may be naturally occurring single domain antibodies known as heavy chain antibody devoid of light chains. In particular, Camelidae species, for example camel, dromedary, llama, alpaca and guanaco, produce heavy chain antibodies naturally devoid of light chain. Camelid heavy chain antibodies also lack the CH1 domain.

The variable heavy chain of these single domain antibodies devoid of light chains are known in the art as “VHH” or “nanobody”. Similar to conventional VH domains, VHHs contain four FRs and three CDRs. Nanobodies have advantages over conventional antibodies: they are about ten times smaller than IgG molecules, and as a consequence properly folded functional nanobodies can be produced by in vitro expression while achieving high yield. Furthermore, nanobodies are very stable, and resistant to the action of proteases. The properties and production of nanobodies have been reviewed by Harmsen and De Haard (Harmsen and De Haard (2007) Appl. Microbiol. Biotechnol. 77:13-22).

The term “monoclonal antibody” or “mAb” as used herein refers to an antibody molecule of a single amino acid composition that is directed against a specific antigen, and is not to be construed as requiring production of the antibody by any particular method. A monoclonal antibody may be produced by a single clone of B cells or hybridoma, but may also be recombinant, i.e. produced by protein engineering.

The term “chimeric antibody” refers to an engineered antibody which in its broadest sense contains one or more region(s) from one antibody and one or more regions from one or more other antibody(ies). In particular a chimeric antibody comprises a VH domain and a VL domain of an antibody derived from a non-human animal, in association with a CH domain and a CL domain of another antibody, in particular a human antibody. As the non-human animal, any animal such as mouse, rat, hamster, rabbit or the like can be used. A chimeric antibody may also denote a multispecific antibody having specificity for at least two different antigens. In an embodiment, a chimeric antibody has variable domains of mouse origin and constant domains of human origin.

The term “humanised antibody” refers to an antibody which is initially wholly or partially of non-human origin and which has been modified to replace certain amino acids, in particular in the framework regions of the heavy and light chains, in order to avoid or minimize an immune response in humans. The constant domains of a humanized antibody are most of the time human CH and CL domains. In an embodiment, a humanized antibody has constant domains of human origin.

“Fragments” of (conventional) antibodies comprise a portion of an intact antibody, in particular the antigen binding region or variable region of the intact antibody. Examples of antibody fragments include Fv, Fab, F(ab′)₂, Fab′, dsFv, (dsFv)₂, scFv, sc(Fv)₂, diabodies, bispecific and multispecific antibodies formed from antibody fragments. A fragment of a conventional antibody may also be a single domain antibody, such as a heavy chain antibody or VHH.

The term “Fab” denotes an antibody fragment having a molecular weight of about 50,000 Da and antigen binding activity, in which about a half of the N-terminal side of H chain and the entire L chain, among fragments obtained by treating IgG with a protease, papaine, are bound together through a disulfide bond.

The term “F(ab′)₂” refers to an antibody fragment having a molecular weight of about 100,000 Da and antigen binding activity, which is slightly larger than the Fab bound via a disulfide bond of the hinge region, among fragments obtained by treating IgG with a protease, pepsin.

The term “Fab” refers to an antibody fragment having a molecular weight of about 50,000 Da and antigen binding activity, which is obtained by cutting a disulfide bond of the hinge region of the F(ab′)₂ fragment.

A single chain Fv (“scFv”) polypeptide is a covalently linked VH::VL heterodimer which is usually expressed from a gene fusion including VH and VL encoding genes linked by a peptide-encoding linker. The human scFv fragment of the invention includes CDRs that are held in appropriate conformation, in particular by using gene recombination techniques. Divalent and multivalent antibody fragments can form either spontaneously by association of monovalent scFvs, or can be generated by coupling monovalent scFvs by a peptide linker, such as divalent sc(Fv)₂.

“dsFv” is a VH::VL heterodimer stabilised by a disulphide bond.

“(dsFv)₂” denotes two dsFv coupled by a peptide linker.

The term “bispecific antibody” or “BsAb” denotes an antibody which combines the antigen-binding sites of two antibodies within a single molecule. Thus, BsAbs are able to bind two different antigens simultaneously. Genetic engineering has been used with increasing frequency to design, modify, and produce antibodies or antibody derivatives with a desired set of binding properties and effector functions as described for instance in EP 2 050 764 A1.

The term “multispecific antibody” denotes an antibody which combines the antigen-binding sites of two or more antibodies within a single molecule.

The term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites.

In a particular embodiment, the epitope-binding fragment is selected from the group consisting of Fv, Fab, F(ab′)₂, Fab′, dsFv, (dsFv)₂, scFv, sc(Fv)₂, diabodies and VHH.

In a particular embodiment, the conjugate of the invention comprises an antibody or epitope-binding fragment thereof which comprises one or more CDR(s) having an amino acid sequence selected from the group consisting of SYNMH (SEQ ID NO: 1), YIYPGNGATNYNQKFKG (SEQ ID NO: 2), GDSVPFAY (SEQ ID NO: 3), SAHSSVSFMH (SEQ ID NO: 4), STSSLAS (SEQ ID NO: 5) and QQRSSFPLT (SEQ ID NO: 6).

In a further embodiment, the conjugate of the invention may comprise an antibody or epitope-binding fragment thereof which comprises a CDR1-H of sequence SEQ ID NO: 1, a CDR2-H of sequence SEQ ID NO: 2 and a CDR3-H of sequence SEQ ID NO: 3.

In a further embodiment, the conjugate of the invention may comprise an antibody or epitope-binding fragment thereof which comprises a CDR1-L of sequence SEQ ID NO: 4, a CDR2-L of sequence SEQ ID NO: 5 and a CDR3-L of sequence SEQ ID NO: 6.

In a further embodiment, the conjugate of the invention may comprise an antibody or epitope-binding fragment thereof which comprises a CDR1-H of sequence SEQ ID NO: 1, a CDR2-H of sequence SEQ ID NO: 2, a CDR3-H of sequence SEQ ID NO: 3, a CDR1-L of sequence SEQ ID NO: 4, a CDR2-L of sequence SEQ ID NO: 5 and a CDR3-L of sequence SEQ ID NO: 6.

Also provided is a conjugate which comprises an antibody or epitope-binding fragment which comprises a heavy chain variable region of sequence

(SEQ ID NO: 7)
QAQLVQSGAEVVKPGASVKMSCKASGYTFTSYNMHWVKQTPGQGLEWIGY
IYPGNGATNYNQKFQGKATLTADPSSSTAYMQISSLTSEDSAVYFCARGD
SVPFAYWGQGTLVTVSA

or a sequence at least 85%, more particularly at least 90%, 91%, 92%, 93%, 94%, 95%, 96%, 97%, 98%, 99% or 100% identical thereto, preferably provided that said sequence contains the sequences SEQ ID NO: 1, SEQ ID NO: 2 and SEQ ID NO: 3.

Still provided is a conjugate which comprises an antibody or epitope-binding fragment which comprises a light chain variable region of sequence

(SEQ ID NO: 8)
EIVLTQSPATMSASPGERVTITCSAHSSVSFMHWFQQKPGTSPKLWIYST
SSLASGVPARFGGSGSGTSYSLTISSMEAEDAATYYCQQRSSFPLTFGAG
TKLELKR

or a sequence at least 85%, more particularly at least 90%, 91%, 92%, 93%, 94%, 95%, 96%, 97%, 98%, 99% or 100% identical thereto, preferably provided that said sequence contains the sequences SEQ ID NO: 4, SEQ ID NO: 5 and SEQ ID NO: 6.

Still provided is a conjugate which comprises an antibody or epitope-binding fragment which comprises a heavy chain of sequence

(SEQ ID NO: 9)
QAQLVQSGAEVVKPGASVKMSCKASGYTFTSYNMHWVKQTPGQGLEWIGY
IYPGNGATNYNQKFQGKATLTADPSSSTAYMQISSLTSEDSAVYFCARGD
SVPFAYWGQGTLVTVSAASTKGPSVFPLAPSSKSTSGGTAALGCLVKDYF
PEPVTVSWNSGALTSGVHTFPAVLQSSGLYSLSSVVTVPSSSLGTQTYIC
NVNHKPSNTKVDKKVEPKSCDKTHTCPPCPAPELLGGPSVFLFPPKPKDT
LMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYT
LPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGK,

or a sequence at least 85%, more particularly at least 90%, 91%, 92%, 93%, 94%, 95%, 96%, 97%, 98%, 99% or 100% identical thereto, preferably provided that said sequence contains the sequences SEQ ID NO: 1, SEQ ID NO: 2 and SEQ ID NO: 3.

Still provided is a conjugate which comprises an antibody or epitope-binding fragment which comprises a light chain of sequence

(SEQ ID NO: 10)
EIVLTQSPATMSASPGERVTITCSAHSSVSFMHWFQQKPGTSPKLWIYST
SSLASGVPARFGGSGSGTSYSLTISSMEAEDAATYYCQQRSSFPLTFGAG
TKLELKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC

or a sequence at least 85%, more particularly at least 90%, 91%, 92%, 93%, 94%, 95%, 96%, 97%, 98%, 99% or 100% identical thereto, preferably provided that said sequence contains the sequences SEQ ID NO: 4, SEQ ID NO: 5 and SEQ ID NO: 6.

In another embodiment, humanized anti-MUC1 antibodies and epitope-binding fragments thereof are provided having a humanized or resurfaced heavy chain variable region having an amino acid sequence corresponding to SEQ ID NO: 7.

Similarly, humanized anti-MUC1 antibodies and epitope-binding fragments thereof are provided having a humanized or resurfaced light chain variable region having an amino acid sequence corresponding to SEQ ID NO: 8.

As used herein, the term “humanized antibody” refers to a chimeric antibody which contain minimal sequence derived from non-human immunoglobulin.

A “chimeric antibody”, as used herein, is an antibody in which the constant region, or a portion thereof, is altered, replaced, or exchanged, so that the variable region is linked to a constant region of a different species, or belonging to another antibody class or subclass. “Chimeric antibody” also refers to an antibody in which the variable region, or a portion thereof, is altered, replaced, or exchanged, so that the constant region is linked to a variable region of a different species, or belonging to another antibody class or subclass.

The goal of humanization is a reduction in the immunogenicity of a xenogenic antibody, such as a murine antibody, for introduction into a human, while maintaining the full antigen binding affinity and specificity of the antibody. Humanized antibodies, or antibodies adapted for non-rejection by other mammals, may be produced using several technologies such as resurfacing and CDR grafting. As used herein, the resurfacing technology uses a combination of molecular modeling, statistical analysis and mutagenesis to alter the non-CDR surfaces of antibody variable regions to resemble the surfaces of known antibodies of the target host.

Strategies and methods for the resurfacing of antibodies, and other methods for reducing immunogenicity of antibodies within a different host, are disclosed in U.S. Pat. No. 5,639,641. Briefly, in a particular method, (1) position alignments of a pool of antibody heavy and light chain variable regions is generated to give a set of heavy and light chain variable region framework surface exposed positions wherein the alignment positions for all variable regions are at least about 98% identical; (2) a set of heavy and light chain variable region framework surface exposed amino acid residues is defined for a rodent antibody (or fragment thereof); (3) a set of heavy and light chain variable region framework surface exposed amino acid residues that is most closely identical to the set of rodent surface exposed amino acid residues is identified; (4) the set of heavy and light chain variable region framework surface exposed amino acid residues defined in step (2) is substituted with the set of heavy and light chain variable region framework surface exposed amino acid residues identified in step (3), except for those amino acid residues that are within 5 Å of any atom of any residue of the complementarity-determining regions of the rodent antibody; and (5) the humanized rodent antibody having binding specificity is produced.

Antibodies can be humanized using a variety of other techniques including CDR-grafting (EP0239400; WO91/09967; U.S. Pat. Nos. 5,530,101 and 5,585,089), veneering or resurfacing (EP0592106; EP0519596; Padlan (1991) Molecular Immunology 28(4/5):489-498; Studnicka et al. (1994) Protein Engineering 7(6):805-814; Roguska et al. (1994) Proc. Natl. Acad. Sci U.S.A. 91:969-973), and chain shuffling (U.S. Pat. No. 5,565,332). Human antibodies can be made by a variety of methods known in the art including phage display methods. See also U.S. Pat. Nos. 4,444,887, 4,716,111, 5,545,806, and 5,814,318; and International patent application WO98/46645, WO98/50433, WO98/24893, WO98/16654, WO96/34096, WO96/33735, and WO91/10741.

An embodiment of such a humanized antibody is a humanized huDS6 antibody comprising a heavy chain of sequence SEQ ID NO: 9 and a light chain of sequence SEQ ID NO: 10, or an epitope-binding fragment thereof, or a sequence at least 85%, more particularly at least 90%, 91%, 92%, 93%, 94%, 95%, 96%, 97%, 98%, 99% or 100% identical thereto, preferably provided that said sequence contains the sequences SEQ ID NO: 1, SEQ ID NO: 2, SEQ ID NO: 3, SEQ ID NO: 4, SEQ ID NO: 5 and SEQ ID NO: 6.

### Cytotoxic Agent

The term “cytotoxic agent” as used herein refers to a substance that reduces or blocks the function or growth, of cells and/or causes destruction of cells. Accordingly, the cytotoxic agent used in the conjugate of the present invention may be any compound that results on the death of a cell, or induces cell death, or in some manner decreases cell viability. Examples of cytotoxic agents include maytansinoids and maytansinoids analogs, a prodrug, tomamycin derivatives, toxoids, a leptomycin derivative, CC-1065 and CC-1065 analogs, as defined below.

Among the cytotoxic agents that may be used in the present invention to form a conjugate, are maytansinoids and maytansinoid analogs. Examples of suitable maytansinoids include maytansinol and maytansinol analogs. Maytansinoids are drugs that inhibit microtubule formation and that are highly toxic to mammalian cells.

Examples of suitable maytansinol analogues include those having a modified aromatic ring and those having modifications at other positions. Such suitable maytansinoids are disclosed in U.S. Pat. Nos. 4,424,219; 4,256,746; 4,294,757; 4,307,016; 4,313,946; 4,315,929; 4,331,598; 4,361,650; 4,362,663; 4,364,866; 4,450,254; 4,322,348; 4,371,533; 6,333,410; 5,475,092; 5,585,499; and 5,846,545.

Specific examples of suitable analogues of maytansinol having a modified aromatic ring include:
(1) C-19-dechloro (U.S. Pat. No. 4,256,746), prepared by LAH reduction of ansamytocin P2;
(2) C-20-hydroxy (or C-20-demethyl)+/−C-19-dechloro (U.S. Pat. Nos. 4,361,650 and 4,307,016), prepared by demethylation using Streptomyces or Actinomyces or dechlorination using LAH; and
(3) C-20-demethoxy, C-20-acyloxy (—OCOR), +/−dechloro (U.S. Pat. No. 4,294,757), prepared by acylation using acyl chlorides.

Specific examples of suitable analogues of maytansinol having modifications of other positions include:
(1) C-9-SH (U.S. Pat. No. 4,424,219), prepared by the reaction of maytansinol with H₂S or P₂S₅;
(2) C-14-alkoxymethyl (demethoxy/CH₂OR) (U.S. Pat. No. 4,331,598);
(3) C-14-hydroxymethyl or acyloxymethyl (CH₂OH or CH₂OAc) (U.S. Pat. No. 4,450,254), prepared from Nocardia;
(4) C-15-hydroxy/acyloxy (U.S. Pat. No. 4,364,866), prepared by the conversion of maytansinol by Streptomyces;
(5) C-15-methoxy (U.S. Pat. Nos. 4,313,946 and 4,315,929), isolated from Trewia nudiflora;
(6) C-18-N-demethyl (U.S. Pat. Nos. 4,362,663 and 4,322,348), prepared by the demethylation of maytansinol by Streptomyces; and
(7) 4,5-deoxy (U.S. Pat. No. 4,371,533), prepared by the titanium trichloride/LAH reduction of maytansinol.

In a particular embodiment, the conjugates of the present invention utilize the thiol-containing maytansinoid DM1, formally termed N^{2′}-deacetyl-N^{2′}-(3-mercapto-1-oxopropyl)-maytansine, as the cytotoxic agent. DM1 is represented by the following structural formula (I):

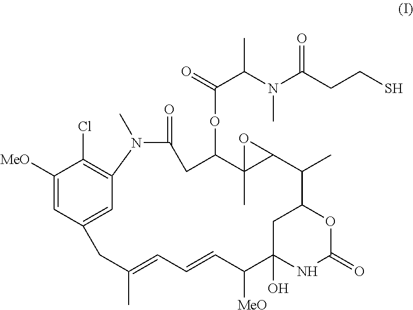 [SMILES: COc1cc2cc(c1Cl)N(C)C(=O)CC(OC(=O)C(C)N(C)C(=O)CCS)C1(C)OC1C(C)C1CC(O)(NC(=O)O1)C(OC)/C=C/C=C(\C)C2]

In another embodiment, the conjugates of the present invention utilize the thiol-containing maytansinoid DM4, formally termed N^{2′}-deacetyl-N^{2′}-(4-methyl-4-mercapto-1-oxopentyl)-maytansine, as the cytotoxic agent. DM4 is represented by the following structural formula (II):

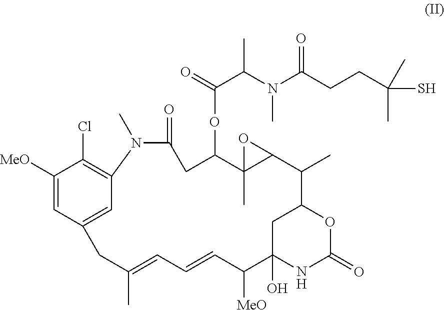 [SMILES: COc1cc2cc(c1Cl)N(C)C(=O)CC(OC(=O)C(C)N(C)C(=O)CCC(C)(C)S)C1(C)OC1C(C)C1CC(O)(NC(=O)O1)C(OC)/C=C/C=C(\C)C2]

In further embodiments of the invention, other maytansines, including thiol and disulfide-containing maytansinoids bearing a mono or di-alkyl substitution on the carbon atom bearing the sulfur atom, may be used. These include a maytansinoid having, at C-3, C-14 hydroxymethyl, C-15 hydroxy, or C-20 desmethyl, an acylated amino acid side chain with an acyl group bearing a hindered sulfhydryl group, wherein the carbon atom of the acyl group bearing the thiol functionality has one or two substituents, said substituents being CH₃, C₂H₅, linear or branched alkyl or alkenyl having from 1 to 10 carbon atoms, cyclic alkyl or alkenyl having from 3 to 10 carbon atoms, phenyl, substituted phenyl, or heterocyclic aromatic or heterocycloalkyl radical, and further wherein one of the substituents can be H, and wherein the acyl group has a linear chain length of at least three carbon atoms between the carbonyl functionality and the sulphur atom.

Such additional maytansines include compounds represented by formula (III):

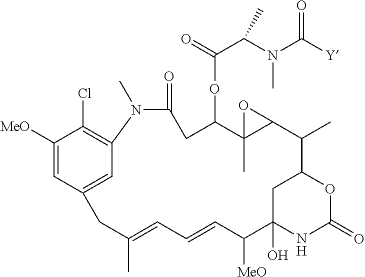

wherein:

Y′ represents

(CR₇R₈)ₗ(CR₉═CR₁₀)ₚ(C≡C)_{q}Aᵣ(CR₅R₆)ₘDᵤ(CR₁₁═CR₁₂)ᵣ(C≡C)ₛBₜ(CR₃R₄)ₙCR₁R₂SZ,
wherein
R₁ and R₂ are each independently CH₃, C₂H₅, linear alkyl or alkenyl having from 1 to 10 carbon atoms, branched or cyclic alkyl or alkenyl having from 3 to 10 carbon atoms, phenyl, substituted phenyl or heterocyclic aromatic or heterocycloalkyl radical, and in addition R₂ can be H;
A, B, D are cycloalkyl or cycloalkenyl having 3-10 carbon atoms, simple or substituted aryl or heterocyclic aromatic or heterocycloalkyl radical; R₃, R₄, R₅, R₆, R₇, R₈, R₉, R₁₀, R₁₁ and R₁₂ are each independently H, CH₃, C₂H₅, linear alkyl or alkenyl having from 1 to 10 carbon atoms, branched or cyclic alkyl or alkenyl having from 3 to 10 carbon atoms, phenyl, substituted phenyl or heterocyclic aromatic or heterocycloalkyl radical;
l, m, n, o, p, q, r, s and t are each independently 0 or an integer of from 1 to 5, provided that at least two of l, m, n, o, p, q, r, s and t are not zero at any one time; and
Z is H, SR or —COR, wherein R is linear alkyl or alkenyl having from 1 to 10 carbon atoms, branched or cyclic alkyl or alkenyl having from 3 to 10 carbon atoms, or simple or substituted aryl or heterocyclic aromatic or heterocycloalkyl radical.

Preferred embodiments of formula (III) include compounds of formula (III) wherein:
R₁ is methyl, R₂ is H and Z is H; R₁ and R₂ are methyl and Z is H;
R₁ is methyl, R₂ is H and Z is —SCH₃;
R₁ and R₂ are methyl and Z is —SCH₃.

Such additional maytansines also include compounds represented by formula (IV-L), (IV-D) or (IV-D,L):

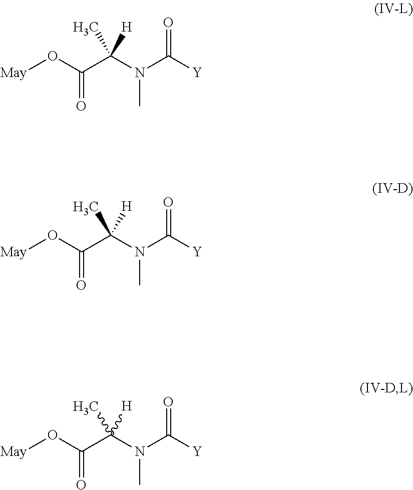

wherein:
Y represents (CR₇R₈)ₗ(CR₅R₆)ₘ(CR₃R₄)ₙCR₁R₂SZ,
wherein:
   R₁ and R₂ are each independently CH₃, C₂H₅, linear alkyl or akenyl having from 1 to 10 carbon atoms, branched or cyclic alkyl or alkenyl having from 3 to 10 carbon atoms, phenyl, substituted phenyl, or heterocyclic aromatic or heterocycloalkyl radical, and in addition R₂ can be H;
   R₃, R₄, R₅, R₆, R₇ and R₈ are each independently H, CH₃, C₂H₅, linear alkyl or alkenyl having from 1 to 10 carbon atoms, branched or cyclic alkyl or alkenyl having from 3 to 10 carbon atoms, phenyl, substituted phenyl, or heterocyclic aromatic or heterocycloalkyl radical;
   l, m and n are each independently an integer of from 1 to 5, and in addition n can be 0;
   Z is H, SR, —COR wherein R is linear or branched alkyl or alkenyl having from 1 to 10 carbon atoms, cyclic alkyl or alkenyl having from 3 to 10 carbon atoms, or simple or substituted aryl or heterocyclic aromatic or heterocyclic radical; and
   May represents a maytansinoid which bears the side chain at C-3, C-14 hydroxymethyl, C-15 hydroxy or C-20 desmethyl.

Particular embodiments of formulae (IV-L), (IV-D) and (IV-D,L) include compounds of formulae (IV-L), (IV-D) and (IV-D,L) wherein:
R₁ is methyl, R₂ is H, R₅, R₆, R₇ and R₈ are each H, l and m are each 1, n is 0, and Z is H;
R₁ and R₂ are methyl, R₅, R₆, R₇ and R₈ are each H, l and m are each 1, n is 0, and Z is H;
R₁ is methyl, R₂ is H, R₅, R₆, R₇ and R₈ are each H, l and m are each 1, n is 0, and Z is —SCH₃;
R₁ and R₂ are methyl, R₅, R₆, R₇ and R₈ are each H, l and m are each 1, n is 0, and Z is —SCH₃.

In one embodiment, the cytotoxic agent is represented by formula (IV-L).

Such additional maytansines also include compounds represented by formula (V):

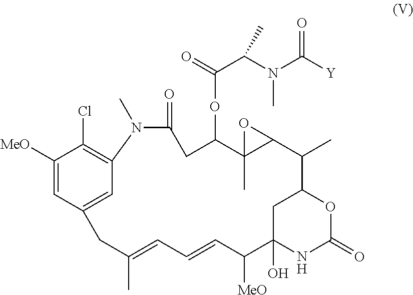

wherein:

Y represents (CR₇R₈)ₗ(CR₅R₆)ₘ(CR₃R₄)ₙCR₁R₂SZ,

wherein:
R₁ and R₂ are each independently CH₃, C₂H₅, linear alkyl or alkenyl having from 1 to 10 carbon atoms, branched or cyclic alkyl or alkenyl having from 3 to 10 carbon atoms, phenyl, substituted phenyl or heterocyclic aromatic or heterocycloalkyl radical, and in addition R₂ can be H;
R₃, R₄, R₅, R₆, R₇ and R₈ are each independently h, CH₃, C₂H₅, linear alkyl or alkenyl having from 1 to 10 carbon atoms, branched or cyclic alkyl or alkenyl having from 3 to 10 carbon atoms, phenyl, substituted phenyl, or heterocyclic aromatic or heterocycloalkyl radical;
l, m and n are each independently an integer of from 1 to 5, and in addition n can be 0; and
Z is H, SR or —COR, wherein R is linear alkyl or alkenyl having from 1 to 10 carbon atoms, branched or cyclic alkyl or alkenyl having from 3 to 10 carbon atoms, or simple or substituted aryl or heterocyclic aromatic or heterocycloalkyl radical.

Particular embodiments of formula (V) include compounds of formula (V) wherein:
R₁ is methyl, R₂ is H, R₅, R₆, R₇ and R₈ are each H, l and m are each 1, n is 0 and Z is H;
R₁ and R₂ are methyl, R₅, R₆, R₇ and R₈ are each H, l and m are 1, n is 0 and Z is H;
R₁ is methyl, R₂ is H, R₅, R₆, R₇ and R₈ are each H, l and m are each 1, n is 0 and Z is —SCH₃;
R₁ and R₂ are methyl, R₅, R₆, R₇ and R₈ are each H, l and m are 1, n is 0 and Z is —SCH₃.

Such additional maytansines further include compounds represented by formula (VI-L), (VI-D) or (VI-D,L):

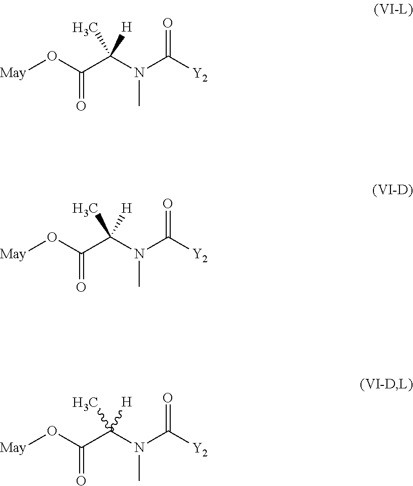

wherein:

Y₂ represents (CR₇R₈)ₗ(CR₅R₆)ₘ(CR₃R₄)ₙCR₁R₂SZ₂,

wherein:
R₁ and R₂ are each independently CH₃, C₂H₅, linear alkyl or alkenyl having from 1 to 10 carbon atoms, branched or cyclic alkyl or alkenyl having from 3 to 10 carbon atoms, phenyl, substituted phenyl or heterocyclic aromatic or heterocycloalkyl radical, and in addition R₂ can be H;
R₃, R₄, R₅, R₆, R₇ and R₈ are each independently H, CH₃, C₂H₅, linear cyclic alkyl or alkenyl having from 1 to 10 carbon atoms, branched or cyclic alkyl or alkenyl having from 3 to 10 carbon atoms, phenyl, substituted phenyl or heterocyclic aromatic or heterocycloalkyl radical;
l, m and n are each independently an integer of from 1 to 5, and in addition n can be 0;
Z₂ is SR or COR, wherein R is linear alkyl or alkenyl having from 1 to 10 carbon atoms, branched or cyclic alkyl or alkenyl having from 3 to 10 carbon atoms, or simple or substituted aryl or heterocyclic aromatic or heterocycloalkyl radical; and
May is a maytansinoid.

Such additional maytansines also include compounds represented by formula (VII):

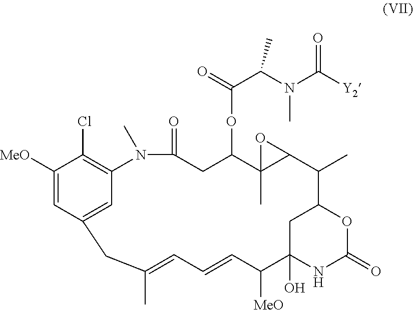

wherein:

Y₂′ represents

(CR₇R₈)ₗ(CR₉═CR₁₀)ₚ(C≡C)_{q}Aᵣ(CR₅R₆)ₘDᵤ(CR₁₁═CR₁₂)ᵣ(C≡C)ₛBₜ(CR₃R₄)ₙCR₁R₂SZ₂,

wherein:
R₁ and R₂ are each independently CH₃, C₂H₅, linear branched or alkyl or alkenyl having from 1 to 10 carbon atoms, cyclic alkyl or alkenyl having from 3 to 10 carbon atoms, phenyl, substituted phenyl or heterocyclic aromatic or heterocycloalkyl radical, and in addition R₂ can be H;
A, B and D are each independently cycloalkyl or cycloalkenyl having 3 to 10 carbon atoms, simple or substituted aryl, or heterocyclic aromatic or heterocycloalkyl radical;
R₃, R₄, R₅, R₆, R₇, R₈, R₉, R₁₀, R₁₁ and R₁₂ are each independently H, CH₃, C₂H₅, linear alkyl or alkenyl having from 1 to 10 carbon atoms, branched or cyclic alkyl or alkenyl having from 3 to 10 carbon atoms, phenyl, substituted phenyl or heterocyclic aromatic or heterocycloalkyl radical;
L, m, n, o, p, q, r, s and t are each independently 0 or an integer of from 1 to 5, provided that at least two of l, m, n, o, p, q, r, s and t are not zero at any one time; and
Z₂ is SR or —COR, wherein R is linear alkyl or alkenyl having from 1 to 10 carbon atoms, branched or cyclic alkyl or alkenyl having from 3 to 10 carbon atoms, or simple or substituted aryl or heterocyclic aromatic or heterocycloalkyl radical.

Particular embodiments of formula (VII) include compounds of formula (VII) wherein R₁ is methyl and R₂ is H.

The above-mentioned maytansinoids can be conjugated to the cell binding agent defined in the section “Cell binding agent” above, in particular to the humanized antibody huDS6 comprising a heavy chain of sequence SEQ ID NO: 9 and a light chain of sequence SEQ ID NO: 10, wherein the cell binding agent, in particular the humanized huDS6 antibody comprising a heavy chain of sequence SEQ ID NO: 9 and a light chain of sequence SEQ ID NO: 10, is linked to the maytansinoid using the thiol or disulfide functionality that is present on the acyl group of an acylated amino acid chain found at C-3, C-14 hydroxymathyl, C-15 hydroxy or C-20 desmethyl of the maytansinoid, and wherein the acyl group of the acylated amino acid side chain has its thiol or disulfide functionality located at a carbon atom that has one or two substituents, said substituents being CH₃, C₂H₅, linear alkyl or alkenyl having from 1 to 10 carbon atoms, branched or cyclic alkyl or alkenyl having from 3 to 10 carbon atoms, phenyl, substituted phenyl or heterocyclic aromatic or heterocycloalkyl radical, and in addition one of the substituents can be H, and wherein the acyl group has a linear chain length of at least three carbon atoms between the carbonyl functionality and the sulfur atom.

In one embodiment of the present invention, the conjugate is the one that comprises the cell binding agent as defined in the section “Cell binding agent” above, in particular the humanized huDS6 antibody comprising a heavy chain of sequence SEQ ID NO: 9 and a light chain of sequence SEQ ID NO: 10, conjugated to a maytansinoid of formula (VIII):

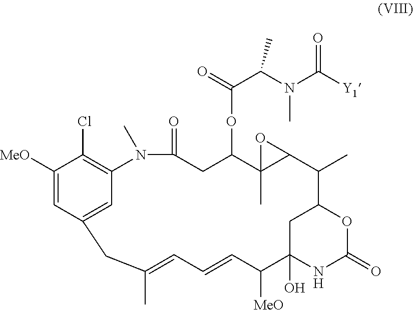

wherein:

Y₁′ represents

(CR₇R₈)ₗ(CR₉═CR₁₀)ₚ(C≡C)_{q}Aᵣ(CR₅R₆)ₘDᵤ(CR₁₁═CR₁₂)ᵣ(C≡C)ₛBₜ(CR₃R₄)ₙCR₁R₂S—,

wherein
A, B and D are each independently cycloalkyl or cycloalkenyl having 3-10 carbon atoms, simple or substituted aryl, or heterocyclic aromatic or heterocycloalkyl radical;
R₃, R₄, R₅, R₆, R₇, R₈, R₉, R₁₀, R₁₁ and R₁₂ are each independently H, CH₃, C₂H₅, linear alkyl or alkenyl having from 1 to 10 carbon atoms, branched or cyclic alkyl or alkenyl having from 3 to 10 carbon atoms, phenyl, substituted phenyl or heterocyclic aromatic or heterocycloalkyl radical; and
l, m, n, o, p, q, r, q and t are each independently 0 or an integer of from 1 to 5, provided that at least two of l, m, n, o, p, q, r, s and t are not zero at any one time.

In particular, R₁ is methyl, R₂ is H, or R₁ and R₂ are methyl.

In a further embodiment of the present invention, the conjugate is the one that comprises the cell binding agent as defined in the section “Cell binding agent” above, in particular the humanized huDS6 antibody comprising a heavy chain of sequence SEQ ID NO: 9 and a light chain of sequence SEQ ID NO: 10, conjugated to a maytansinoid of formula (IX-L), (IX-D) or (IX-D,L):

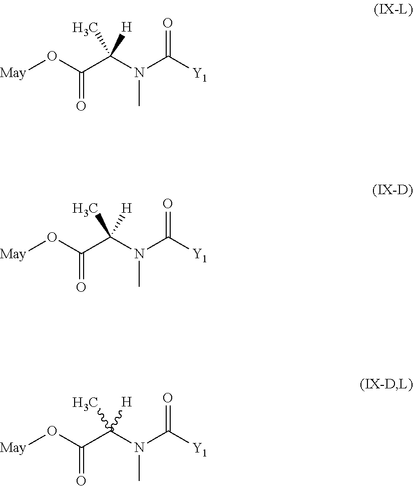

wherein:

Y₁ represents (CR₇R₈)ₗ(CR₅R₆)ₘ(CR₃R₄)ₙCR₁R₂S—,

wherein
R₁ and R₂ are each independently CH₃, C₂H₅, linear alkyl or alkenyl having from 1 to 10 carbon atoms, branched or cyclic alkyl or alkenyl having from 3 to 10 carbon atoms, phenyl, substituted phenyl, heterocyclic aromatic or heterocycloakenyl radical, and in addition R₂ can be H;
R₃, R₄, R₅, R₆, R₇ and R₈ are each independently H, CH₃, C₂H₅, linear alkyl or alkenyl having from 1 to 10 carbon atoms, branched or cyclic alkyl or alkenyl having from 3 to 10 carbon atoms, phenyl, substituted phenyl or heterocyclic aromatic or heterocycloalkyl radical;
l, m and n are each independently an integer from 1 to 5, and in addition n can be 0; and
May represents a maytansinol which bears the side chain at C-3, C-14 hydroxymethyl, C-15 hydroxy or C-20 desmethyl.

Particular embodiments of formulae (IX-L), (IX-D) and (IX-D,L) include compounds of formulae (IX-L), (IX-D) and (IX-D,L) wherein:

R₁ is methyl and R₂ is H, or R₁ and R₂ are methyl,

R₁ is methyl, R₂ is H, R₅, R₆, R₇ and R₈ are each H, l and m are each 1, and n is 0,

R₁ and R₂ are methyl, R₅, R₆, R₇ and R₈ are each H, l and m are each 1, and n is 0.

More particularly, the cytotoxic agent is represented by formula (IX-L).

In a further embodiment of the present invention, the conjugate is the one that comprises the cell binding agent as defined in the section “Cell binding agent” above, in particular the humanized huDS6 antibody comprising a heavy chain of sequence SEQ ID NO: 9 and a light chain of sequence SEQ ID NO: 10, conjugated to a maytansinoid of formula (X):

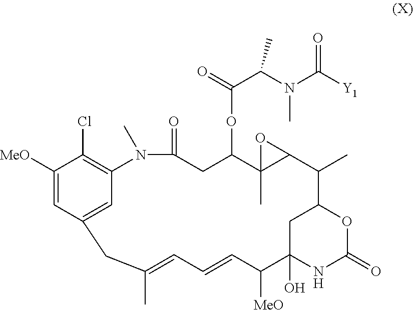

wherein the substituents are as defined for formula (IX) above.

In a further embodiment, in the above-described compounds, R₁ is H, R₂ is methyl, R₅, R₆, R₇ and R₈ are each H, l and m are each 1, and n is 0.

In further embodiments, in the above-described compounds, R₁ and R₂ are methyl, R₅, R₆, R₇ and R₈ are each H, l and m are each 1, and n is 0.

Further, the L-aminoacyl stereoisomer is preferred.

Each of the maytansinoids taught in U.S. patent application Ser. No. 10/849,136 filed May 20, 2004, may also be used as cytotoxic agent in the conjugate of the invention.

Conjugates of cell binding agents as defined in the section “Cell binding agent” above, in particular of antibodies, with maytansinoid drugs can be evaluated for their ability to suppress proliferation of various unwanted cell lines in vitro. For example, cell lines such as the human epidermoid carcinoma line A-431, the human small cell lung cancer cell line SW2, the human breast tumor line SKBR3 and the Burkitt's lymphoma cell line Namalwa can easily be used for the assessment of cytotoxicity of these compounds. Cells to be evaluated can be exposed to the compounds for 24 h and the surviving fractions of cells measured in direct assays by known methods. IC₅₀ values can then be calculated from the results of the assays.

The cytotoxic agent used in the conjugates according to the present invention may also be a taxane or derivative thereof.

Taxanes are a family of compounds that includes paclitaxel (taxol), a cytotoxic natural product, and docetaxel (Taxotere), a semi-synthetic derivative, two compounds that are widely used in the treatment of cancer. Taxanes are mitotic-spindle poisons that inhibit the depolymerization of tubulin, resulting in cell death. While docetaxel and paclitaxel are useful agents in the treatment of cancer, their antitumor activity is limited because of their non-specific toxicity towards normal cells.

A particular taxane for use in the preparation of conjugates is the taxane of formula (XI):

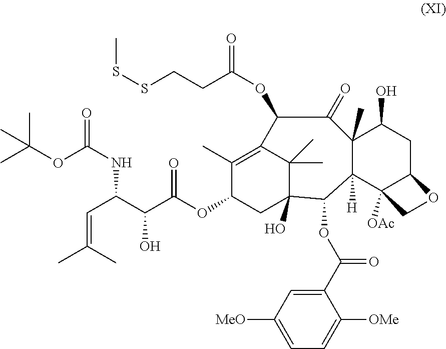 [SMILES: COc1ccc(OC)c(C(=O)O[C@H]2[C@@H]3[C@]4(OC(C)=O)CO[C@@H]4C[C@H](O)[C@@]3(C)C(=O)[C@H](OC(=O)CCSSC)C3=C(C)[C@@H](OC(=O)[C@H](O)[C@H](C=C(C)C)NC(=O)OC(C)(C)C)C[C@]2(O)C3(C)C)c1]

Methods for synthesizing taxanes that may be used in the cytotoxic conjugates of the present invention, along with methods for conjugating the taxanes to a cell binding agent as defined in the section “Cell binding agent” above, such as the humanized huDS6 antibody comprising a heavy chain of sequence SEQ ID NO: 9 and a light chain of sequence SEQ ID NO: 10, are described in detail in U.S. Pat. Nos. 5,416,064, 5,475,092, 6,340,701, 6,372,738 and 6,436,931, and in U.S. application Ser. Nos. 10/024,290, 10/144,042, 10/207,814, 10/210,112 and 10/369,563.

The cytotoxic agent according to the present invention may also be a tomaymycin derivative. Tomaymycin derivatives are pyrrolo[1,4]benzodiazepines (PBDs), a known class of compounds exerting their biological properties by covalently binding to the N2 of guanine in the minor groove of DNA. PBDs include a number of minor groove binders such as anthramycin, neothramycin and DC-81.

Novel tomaymycin derivatives that retain high cytotoxicity and that can be effectively linked to cell binding agents as defined in the section “Cell binding agent” above are described in the International Application No. PCT/IB2007/000142. The cell binding agent-tomaymycin derivative complexes permit the full measure of the cytotoxic action of the tomaymycin derivatives to be applied in a targeted fashion against unwanted cells only, therefore avoiding side effects due to damage to non-targeted healthy cells.

The cytotoxic agent according to the present invention may comprise one or more tomaymycin derivatives, linked to a cell binding agent as defined in the section “Cell binding agent” above, such as the humanized huDS6 antibody comprising a heavy chain of sequence SEQ ID NO: 9 and a light chain of sequence SEQ ID NO: 10, via a linking group. The linking group is part of a chemical moiety that is covalently bound to a tomaymycin derivative through conventional methods. In a particular embodiment, the chemical moiety can be covalently bound to the tomaymycin derivative via a disulfide bond.

The tomaymycin derivatives useful in the present invention have the formula (XII) shown below:

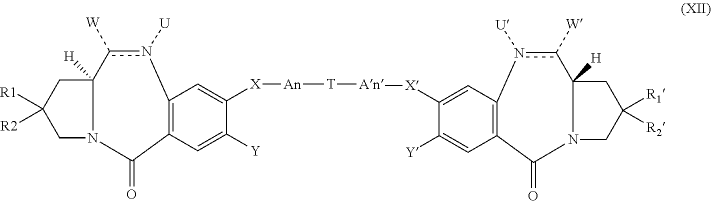

wherein
- - - represents an optional single bond; [symbol]
represents either a single bond or a double bond;
provided that when [symbol] represents a single bond, U and U′, the same or different, independently represent H, and W and W′, the same or different, are independently selected from the group consisting of OH, an ether such as —OR, an ester (e.g. an acetate), such as —OCOR, a carbonate such as —OCOOR, a carbamate such as —OCONRR′, a cyclic carbamate, such that N10 and C11 are a part of the cycle, a urea such as —NRCONRR′, a thiocarbamate such as —OCSNHR, a cyclic thiocarbamate such that N10 and C11 are a part of the cycle, —SH, a sulfide such as —SR, a sulphoxide such as —SOR, a sulfone such as —SOOR, a sulphonate such as —SO3-, a sulfonamide such as —NRSOOR, an amine such as —NRR′, optionally cyclic amine such that N10 and C11 are a part of the cycle, a hydroxylamine derivative such as —NROR′, an amide such as —NRCOR′, an azido such as —N3, a cyano, a halo, a trialkyl or triarylphosphonium, an aminoacid-derived group. Preferably W and W′ are the same or different and are OH, Ome, Oet, NHCONH₂, SMe;
and when [symbol] represents a double bond, U and U′ are absent and W and W′ represent H;

R1, R2, R1′, R2′ are the same or different and independently chosen from Halide or Alkyl optionally substituted by one or more Hal, CN, NRR′, CF₃, OR, Aryl, Het, S(O)_{q}R, or R1 and R2 and R1′ and R2′ form together a double bond containing group ═B and ═B′ respectively.

In one embodiment, R1 and R2 and R1′ and R2′ form together a double bond containing group ═B and ═B′ respectively.

—B and B′ are the same or different and independently chosen from Alkenyl being optionally substituted by one or more Hal, CN, NRR′, CF₃, OR, Aryl, Het, S(O)_{q}R or B and B′ represent an oxygen atom.

In one embodiment, B═B′.

In a further embodiment, B═B′═ ═CH₂ or ═CH—CH₃,

X and X′ are the same or different and independently chosen from one or more —O—, —NR—, —(C═O)—, —S(O)_{q}—.

In one embodiment, X═X′.

In a further embodiment, X═X′═O.

A and A′ are the same or different and independently chosen from Alkyl or Alkenyl optionally containing an oxygen, a nitrogen or a sulfur atom, each being optionally substituted by one or more Hal, CN, NRR′, CF₃, OR, S(O)_{q}R, Aryl, Het, Alkyl, Alkenyl.

In one embodiment, A=A′.

In a further embodiment, A=A′=linear unsubstituted alkyl.

Y and Y′ are the same or different and independently chosen from H, OR;

In one embodiment, Y═Y′.

In a further embodiment, Y═Y′=OAlkyl, more preferably OMethyl.

T is —NR—, —O—, —S(O)_{q}—, or a 4 to 10-membered aryl, cycloalkyl, heterocyclic or heteroaryl, each being optionally substituted by one or more Hal, CN, NRR′, CF₃, R, OR, S(O)_{q}R, and/or linker(s), or a branched Alkyl, optionally substituted by one or more Hal, CN, NRR′, CF₃, OR, S(O)_{q}R and/or linker(s), or a linear Alkyl substituted by one or more Hal, CN, NRR′, CF₃, OR, S(O)_{q}R and/or linker(s).

In one embodiment, T is a 4 to 10-membered aryl or heteroaryl, more preferably phenyl or pyridyl, optionally substituted by one or more linker(s).

Said linker comprises a linking group. Suitable linking groups are well known in the art and include thiol, sulfide, disulfide groups, thioether groups, acid labile groups, photolabile groups, peptidase labile groups and esterase labile groups. Preferred are disulfide groups and thioether groups.

When the linking group is a thiol-, sulfide (or so-called thioether —S—) or disulfide (—S—S—)-containing group, the side chain carrying the thiol, the sulfide or disulfide group can be linear or branched, aromatic or heterocyclic. One of ordinary skill in the art can readily identify suitable side chains.

In one embodiment, said linker is of formula -G-D-(Z)P—S—Z′

where
G is a single or double bond, —O—, —S— or —NR—;
D is a single bond or -E-, -E-NR—, -E-NR—F—, -E-O—, -E-O—F—, -E-NR—CO—, -E-NR—CO—F—, -E-CO—, —CO-E-, -E-CO—F, -E-S—, -E-S—F—, -E-NR—C—S—, -E-NR—CS—F—;
   where E and F are the same or different and are independently chosen from linear or branched —(OCH₂CH₂)ᵢAlkyl(OCH₂CH₂)ⱼ—, -Alkyl(OCH₂CH₂)ᵢ-Alkyl-, —(OCH₂CH₂)ᵢ—, —(OCH₂CH₂)ᵢCycloalkyl(OCH₂CH₂)ⱼ—, (OCH₂CH₂)ᵢHeterocyclic(OCH₂CH₂)ⱼ—, —(OCH₂CH₂)ᵢAryl(OCH₂CH₂)ⱼ—, (OCH₂CH₂)ᵢHeteroaryl(OCH₂CH₂)ⱼ—, -Alkyl-(OCH₂CH₂)ᵢAlkyl(OCH₂CH₂)ⱼ—, -Alkyl-(OCH₂CH₂)ᵢ—, -Alkyl-(OCH₂CH₂)ᵢCycloalkyl(OCH₂CH₂)ⱼ—, Alkyl(OCH₂CH₂)ᵢHeterocyclic(OCH₂CH₂)ⱼ—, -Alkyl-(OCH₂CH₂)ᵢAryl(OCH₂CH₂)ⱼ—, -Alkyl(OCH₂CH₂)ᵢHeteroaryl(OCH₂CH₂)ⱼ—, -Cycloalkyl-Alkyl-, -Alkyl-Cycloalkyl-, -Heterocyclic-Alkyl-, -Alkyl-Heterocyclic-, -Alkyl-Aryl-, -Aryl-Alkyl-, -Alkyl-Heteroaryl-, -Heteroaryl-Alkyl-;
      where i and j, identical or different, are integers and independently chosen from 0, 1 to 2000;
Z is linear or branched -Alkyl-;
p is 0 or 1;
Z′ represents H, a thiol protecting group such as COR, R₂₀ or SR₂₀, wherein R₂₀ represents H, methyl, Alkyl, optionally substituted Cycloalkyl, aryl, heteroaryl or heterocyclic, provided that when Z′ is H, said compound is in equilibrium with the corresponding compound formed by intramolecular cyclisation resulting from addition of the thiol group —SH on the imine bond —NH═ of one of the PBD moieties.
n, n′, equal or different are 0 or 1.
q is 0, 1 or 2.
R and R′ are equal or different and independently chosen from H, Alkyl, Aryl, each being optionally substituted by Hal, CN, NRR′, CF₃, R, OR, S(O)_{q}R, Aryl, Het;
   or their pharmaceutically acceptable salts, hydrates, or hydrated salts, or the polymorphic crystalline structures of these compounds or their optical isomers, racemates, diastereomers or enantiomers.

The compounds of the general formula (XII) having geometrical and stereoisomers are also a part of the invention.

The N-10, C-11 double bond of tomaymycin derivatives of formula (XII) is known to be readily convertible in a reversible manner to corresponding imine adducts in the presence of water, an alcohol, a thiol, a primary or secondary amine, urea and other nucleophiles. This process is reversible and can easily regenerate the corresponding tomaymycin derivatives in the presence of a dehydrating agent, in a non-protic organic solvant, in vacuum or at high temperatures (Tozuka (1983) J. Antibiotics 36:276).

Thus, reversible derivatives of tomaymycin derivatives of general formula (XIII) can also be used in the present invention:

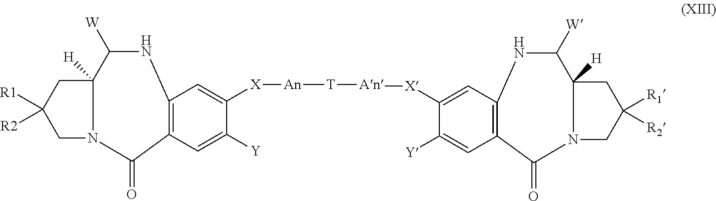

where A, X, Y, n, T, A′, X′, Y′, n′, R1, R2, R1′, R2′ are defined as in formula (XII) and W and W′ are the same or different and are selected from the group consisting of OH, an ether such as —OR, an ester (e.g. an acetate), such as —OCOR, —COOR, a carbonate such as —OCOOR, a carbamate such as —OCONRR′, a cyclic carbamate, such that N10 and C11 are a part of the cycle, a urea such as —NRCONRR′, a thiocarbamate such as —OCSNHR, a cyclic thiocarbamate such that N10 and C11 are a part of the cycle, —SH, a sulfide such as —SR, a sulphoxide such as —SOR, a sulfone such as —SOOR, a sulphonate such as —SO₃—, a sulfonamide such as —NRSOOR, an amine such as —NRR′, optionally cyclic amine such that N10 and C11 are a part of the cycle, a hydroxylamine derivative such as —NROR′, an amide such as —NRCOR, —NRCONRR′, an azido such as —N₃, a cyano, a halo, a trialkyl or triarylphosphonium, an aminoacid-derived group. Preferably, W and W′ are the same or different and are OH, Ome, Oet, NHCONH₂, SMe.

Compounds of formula (XIII) may thus be considered as solvates, including water when the solvent is water; these solvates can be particularly useful.

In a further embodiment, the tomaymycin derivatives of the invention are selected from the group consisting in:
8,8′-[1,3-benzenediylbis(methyleneoxy)]-bis[(S)-2-eth-(E)-ylidene-7-methoxy-1,2,3,11a-tetrahydro-5H-pyrrolo[2,1-c][1,4]benzodiazepin-5-one]
8,8′-[5-methoxy-1,3-benzenediylbis(methyleneoxy)]-bis[(S)-2-eth-(E)-ylidene-7-methoxy-1,2,3,11a-tetrahydro-5H-pyrrolo[2,1-c][1,4]benzodiazepin-5-one]
8,8′-[1,5-pentanediylbis(oxy)]-bis[(S)-2-eth-(E)-ylidene-7-methoxy-1,2,3,11a-tetrahydro-5H-pyrrolo[2,1-c][1,4]benzodiazepin-5-one]
8,8′-[1,4-butanediylbis(oxy)]-bis[(S)-2-eth-(E)-ylidene-7-methoxy-1,2,3,11a-tetrahydro-5H-pyrrolo[2,1-c][1,4]benzodiazepin-5-one]
8,8′-[3-methyl-1,5-pentanediylbis(oxy)]-bis[(S)-2-eth-(E)-ylidene-7-methoxy-1,2,3,11a-tetrahydro-5H-pyrrolo[2,1-c][1,4]benzodiazepin-5-one]
8,8′-[2,6-pyridinediylbis(oxy)]-bis[(S)-2-eth-(E)-ylidene-7-methoxy-1,2,3,11a-tetrahydro-5H-pyrrolo[2,1-c][1,4]benzodiazepin-5-one]
8,8′-[4-(3-tert-butoxycarbonylaminopropyloxy)-2,6-pyridinediylbis-(methyleneoxy)]-bis[(S)-2-eth-(E)-ylidene-7-methoxy-1,2,3,11a-tetrahydro-5H-pyrrolo[2,1-c][1,4]benzodiazepin-5-one]
8,8′-[5-(3-aminopropyloxy)-1,3-benzenediylbis(methyleneoxy)]-bis[(S)-2-eth-(E)-ylidene-7-methoxy-1,2,3,11a-tetrahydro-5H-pyrrolo[2,1-c][1,4]benzodiazepin-5-one]
8,8′-[5-(N-methyl-3-tert-butoxycarbonylaminopropyl)-1,3-benzenediylbis-(methyleneoxy)]-bis[(S)-2-eth-(E)-ylidene-7-methoxy-1,2,3,11a-tetrahydro-5H-pyrrolo[2,1-c][1,4]benzodiazepin-5-one]
8,8′-{5-[3-(4-methyl-4-methyldisulfanyl-pentanoylamino)propyloxy]-1,3-benzenediylbis(methyleneoxy)}-bis[(S)-2-eth-(E)-ylidene-7-methoxy-1,2,3,11a-tetrahydro-5H-pyrrolo[2,1-c][1,4]benzodiazepin-5-one]
8,8′-[5-acetylthiomethyl-1,3-benzenediylbis(methyleneoxy)]-bis[(S)-2-methylene-7-methoxy-1,2,3,11a-tetrahydro-5H-pyrrolo[2,1-c][1,4]benzodiazepin-5-one]
bis-{2-[(S)-2-methylene-7-methoxy-5-oxo-1,3,11a-tetrahydro-5H-pyrrolo[2,1-c][1,4]benzodiazepin-8-yloxy]-ethyl}-carbamic acid tert-butyl ester
8,8′-[3-(2-acetylthioethyl)-1,5-pentanediylbis(oxy)]-bis[(S)-2-methylene-7-methoxy-1,2,3,11a-tetrahydro-5H-pyrrolo[2,1-c][1,4]benzodiazepin-5-one]
8,8′-[5-(N-4-mercapto-4,4-dimethylbutanoyl)amino-1,3-benzenediylbis(methyleneoxy)]-bis[7-methoxy-2-methylene-1,2,3,11a-tetrahydro-5H-pyrrolo[2,1-c][1,4]benzodiazepin-5-one]
8,8′-[5-(N-4-methyldithio-4,4-dimethylbutanoyl)-amino-1,3-benzenediylbis(methyleneoxy)]-bis[7-methoxy-2-methylene-1,2,3,11a-tetrahydro-5H-pyrrolo[2,1-c][1,4]benzodiazepin-5-one]
8,8′-[5-(N-methyl-N-(2-mercapto-2,2-dimethylethyl)amino-1,3-benzenediyl(methyleneoxy)]-bis[7-methoxy-2-methylene-1,2,3,11a-tetrahydro-5H-pyrrolo[2,1-c][1,4]benzodiazepin-5-one]
8,8′-[5-(N-methyl-N-(2-methyldithio-2,2-dimethylethyl)amino-1,3-benzenediyl(methyleneoxy)]-bis[7-methoxy-2-methylene-1,2,3,11a-tetrahydro-5H-pyrrolo[2,1-c][1,4]benzodiazepin-5-one]
8,8′-[(4-(2-(4-mercapto-4-methyl)-pentanamido-ethoxy)-pyridin-2,6-dimethyl)-dioxy]-bis[(S)-2-eth-(E)-ylidene-7-dimethoxy-1,2,3,11a-tetrahydro-pyrrolo[2,1-c][1,4]benzodiazepin-5-one]
8,8′-[(1-(2-(4-methyl-4-methyldisulfanyl)-pentanamido-ethoxy)-benzene-3,5-dimethyl)-dioxy]-bis[(S)-2-eth-(E)-ylidene-7-dimethoxy-1,2,3,11a-tetrahydro-pyrrolo[2,1-c][1,4]benzodiazepin-5-one]
8,8′-[(4-(3-(4-methyl-4-methyldisulfanyl)-pentanamido-propoxy)-pyridin-2,6-dimethyl)-dioxy]-bis[(S)-2-eth-(E)-ylidene-7-dimethoxy-1,2,3,11a-tetrahydro-pyrrolo[2,1-c][1,4]benzodiazepin-5-one]
8,8′-[(4-(4-(4-methyl-4-methyldisulfanyl)-pentanamido-butoxy)-pyridin-2,6-dimethyl)-dioxy]-bis[(S)-2-eth-(E)-ylidene-7-dimethoxy-1,2,3,11a-tetrahydro-pyrrolo[2,1-c][1,4]benzodiazepin-5-one]
8,8′-[(4-(3-[4-(4-methyl-4-methyldisulfanyl-pentanoyl)-piperazin-1-yl]-propyl)-pyridin-2,6-dimethyl)-dioxy]-bis[(S)-2-eth-(E)-ylidene-7-dimethoxy-1,2,3,11a-tetrahydro-pyrrolo[2,1-c][1,4]benzodiazepin-5-one]
8,8′-[(1-(3-[4-(4-methyl-4-methyldisulfanyl-pentanoyl)-piperazin-1-yl]-propyl)-benzene-3,5-dimethyl)-dioxy]-bis[(S)-2-eth-(E)-ylidene-7-dimethoxy-1,2,3,11a-tetrahydro-pyrrolo[2,1-c][1,4]benzodiazepin-5-one]
8,8′-[(4-(2-{2-[2-(4-methyl-4-methyldisulfanyl-pentanoylamino)-ethoxy]-ethoxy}-ethoxy)-pyridin-2,6-dimethyl)-dioxy]-bis[(S)-2-eth-(E)-ylidene-7-di methoxy-1,2,3,11a-tetrahydro-pyrrolo[2,1-c][1,4]benzodiazepin-5-one]
8,8′-[(1-(2-{2-[2-(2-{2-[2-(4-methyl-4-methyldisulfanyl-pentanoylamino)-ethoxy]-ethoxy}-ethoxy)-ethoxy]-ethoxy}-ethoxy)-benzene-3,5-dimethyl)-dioxy]-bis[(S)-2-eth-(E)-ylidene-7-dimethoxy-1,2,3,11a-tetrahydro-pyrrolo[2,1-c][1,4]benzodiazepin-5-one]
8,8′-[(1-(2-{2-[2-(4-methyl-4-methyldisulfanyl-pentanoylamino)-ethoxy]-ethoxy}-ethoxy)-benzene-3,5-dimethyl)-dioxy]-bis[(S)-2-eth-(E)-ylidene-7-di methoxy-1,2,3,11a-tetrahydro-pyrrolo[2,1-c][1,4]benzodiazepin-5-one]
8,8′-[(4-(2-{2-[2-(2-{2-[2-(4-methyl-4-methyldisulfanyl-pentanoylamino)-ethoxy]-ethoxy}-ethoxy)-ethoxy]-ethoxy}-ethoxy)-pyridin-2,6-dimethyl)-dioxy]-bis[(S)-2-eth-(E)-ylidene-7-dimethoxy-1,2,3,11a-tetrahydro-pyrrolo[2,1-c][1,4]benzodiazepin-5-one]
8,8′-[(1-(2-[methyl-(2-methyl-2-methyldisulfanyl-propyl)-amino]-ethoxy)-benzene-3,5-dimethyl)-dioxy]-bis[(S)-2-eth-(E)-ylidene-7-dimethoxy-1,2,3,11a-tetrahydro-pyrrolo[2,1-c][1,4]benzodiazepin-5-one]
8,8′-[(4-(3-[methyl-(4-methyl-4-methyldisulfanyl-pentanoyl)-amino]-propyl)-pyridin-2,6-dimethyl)-dioxy]-bis[(S)-2-eth-(E)-ylidene-7-dimethoxy-1,2,3,11a-tetrahydro-pyrrolo[2,1-c][1,4]benzodiazepin-5-one]
8,8′-[(4-(3-[methyl-(2-methyl-2-methyl disulfanyl-propyl)-amino]-propyl)-pyridin-2,6-dimethyl)-dioxy]-bis[(S)-2-eth-(E)-ylidene-7-dimethoxy-1,2,3,11a-tetrahydro-pyrrolo[2,1-c][1,4]benzodiazepin-5-one]
8,8′-[(1-(4-methyl-4-methyldisulfanyl)-pentanamido)-benzene-3,5-dimethyl)-dioxy]-bis[(S)-2-eth-(E)-ylidene-7-dimethoxy-1,2,3,11a-tetrahydro-pyrrolo[2,1-c][1,4]benzodiazepin-5-one]
   as well as the corresponding mercapto derivatives, or their pharmaceutically acceptable salts, hydrates, or hydrated salts, or the polymorphic crystalline structures of these compounds or their optical isomers, racemates, diastereomers or enantiomers.

Particular compounds are those of formula (XIV) or (XV):

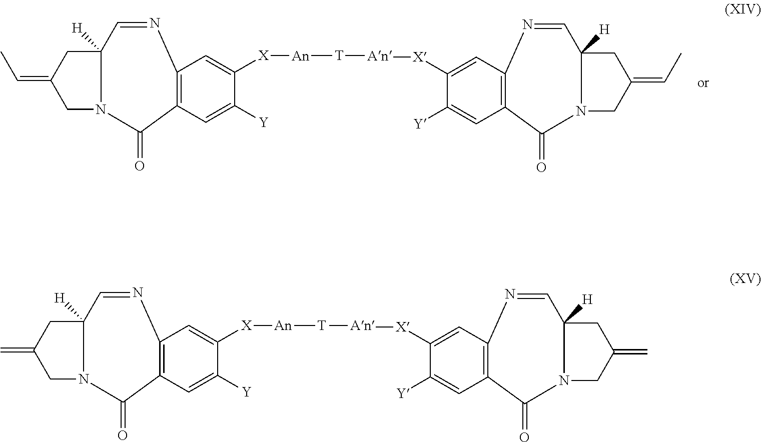

where X, X′, A, A′, Y, Y′, T, n, n′ are defined as in formula (XII).

The compounds of formula (XII) may be prepared in a number of ways well known to those skilled in the art. The compounds can be synthesized, for example, by application or adaptation of the methods described below, or variations thereon as appreciated by the skilled artisan. The appropriate modifications and substitutions will be readily apparent and well known or readily obtainable from the scientific literature to those skilled in the art. In particular, such methods can be found in R. C. Larock, Comprehensive Organic Transformations, Wiley-VCH Publishers, 1999.

Methods for synthesizing the tomaymycin derivatives which may be used in the invention are described in the International Application No. PCT/IB2007/000142. Compounds of the present invention may be prepared by a variety of synthetic routes. The reagents and starting materials are commercially available, or readily synthesized by well-known techniques by one of ordinary skill in the arts (see, for example, WO00/12508, WO00/12507, WO2005/040170, WO2005/085260, FR1516743, Mori et al. (1986) Tetrahedron 42:3793-3806).

The cytotoxic agent according to the present invention may also be a leptomycin derivative.

According to the present invention, “leptomycin derivatives” refer to members of the leptomycin family as defined in Kalesse et al. (2002) Synthesis 8:981-1003, and includes: leptomycins, such as leptomycin A and leptomycin B, callystatins, ratjadones such as ratjadone A and ratjadone B, anguinomycins such as anguinomycin A, B, C, D, kasusamycins, leptolstatin, leptofuranins, such as leptofuranin A, B, C, D. Derivatives of leptomycin A and B are preferred.

More specifically, the leptomycin derivatives may be of formula (XVI):

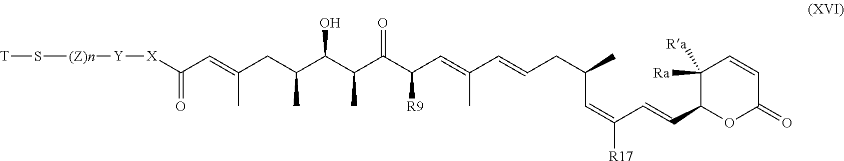

wherein
Ra and Ra′ are H or -Alk; preferably Ra is -Alk, preferably methyl and Ra′ is H;
R17 is alkyl optionally substituted by OR, CN, NRR′, perfluoroalkyl; preferably, R17 is alkyl, more preferably methyl or ethyl;
R9 is alkyl optionally substituted by OR, CN, NRR′, perfluoroalkyl; preferably, R9 is alkyl, more preferably methyl;
X is —O— or —NR—; preferably, X is —NR—;
Y is —U—, —NR—U—, —O—U—, —NR—CO—U—, —U—NR—CO—, —U—CO—, —CO—U—;
preferably, when X is —O—, Y is —U—, —NR—U—, —U—NR—CO—;
   where U is chosen from linear or branched -Alk-, -Alk(OCH₂CH₂)ₘ—, —(OCH₂CH₂)ₘ-Alk-, -Alk(OCH₂CH₂)ₘ-Alk-, —(OCH₂CH₂)ₘ—, -Cycloalkyl-, -Heterocyclic-, -Cycloalkyl-Alk-, -Alk-Cycloalkyl-, -Heterocyclic-Alk-, -Alk-Heterocyclic-;
      where m is an integer chosen from 1 to 2000;
   preferably, U is linear or branched -Alk-,
Z is -Alk-;
n is 0 or 1; preferably n is 0;
T represents H, a thiol protecting group such as Ac, R₁ or SR₁, wherein R₁ represents H, methyl, Alk, Cycloalkyl, optionally substituted aryl or heterocyclic, or T represents

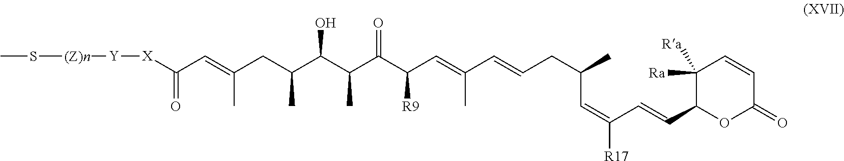

where:
Ra, Ra′, R17, R9, X, Y, Z, n are defined as above;
preferably, T is H or SR₁, wherein R₁ represents Alk, more preferably methyl;
R and R′ identical or different are H or alkyl;
Alk represents a linear or branched alkyl; preferably Alk represents (—(CH_{2-q}(CH₃)_{q})ₚ— where p represents an integer from 1 to 10 and q represents an integer from 0 to 2; preferably, Alk represents —(CH₂)— or —C(CH₃)₂—.
   or their pharmaceutically acceptable salts, hydrates, or hydrated salts, or the polymorphic crystalline structures of these compounds or their optical isomers, racemates, diastereomers or enantiomers.

Particular compounds may be chosen from:
(2-Methylsulfanyl-ethyl)-amid of (2E,10E,12E,16Z,18E)-(R)-6-Hydroxy-3,5,7,9,11,15,17-heptamethyl-19-((2S,3S)-3-methyl-6-oxo-3,6-dihydro-2H-pyran-2-yl)-8-oxo-nonadeca-2,10,12,16,18-pentaenoic acid
Bis-[(2-mercaptoethyl)-amid of (2E,10E,12E,16Z,18E)-(R)-6-hydroxy-3,5,7,9,11,15,17-heptamethyl-19-((2S,3S)-3-methyl-6-oxo-3,6-dihydro-2H-pyran-2-yl)-8-oxo-nonadeca-2,10,12,16,18-pentaenoic acid]
(2-Mercapto-ethyl)-amid of (2E,10E,12E,16Z,18E)-(R)-6-hydroxy-3,5,7,9,11,15,17-heptamethyl-19-((2S,3S)-3-methyl-6-oxo-3,6-dihydro-2H-pyran-2-yl)-8-oxo-nonadeca-2,10,12,16,18-pentaenoic acid
(2-Methyldisulfanyl-ethyl)-amid of (2E,10E,12E,16Z,18E)-(R)-6-hydroxy-3,5,7,9,11,15,17-heptamethyl-19-((2S,3S)-3-methyl-6-oxo-3,6-dihydro-2H-pyran-2-yl)-8-oxo-nonadeca-2,10,12,16,18-pentaenoic acid
(2-Methyl-2-methyldisulfanyl-propyl)-amid of (2E,10E,12E,16Z,18E)-(R)-6-hydroxy-3,5,7,9,11,15,17-heptamethyl-19-((2S,3S)-3-methyl-6-oxo-3,6-dihydro-2H-pyran-2-yl)-8-oxo-nonadeca-2,10,12,16,18-pentaenoic acid
(2-Mercapto-2-methyl-propyl)-amid of (2E,10E,12E,16Z,18E)-(R)-6-hydroxy-3,5,7,9,11,15,17-heptamethyl-19-((2S,3S)-3-methyl-6-oxo-3,6-dihydro-2H-pyran-2-yl)-8-oxo-nonadeca-2,10,12,16,18-pentaenoic acid
   or their pharmaceutically acceptable salts, hydrates, or hydrated salts, or the polymorphic crystalline structures of these compounds or their optical isomers, racemates, diastereomers or enantiomers.

In order to link the derivative to a cell-binding agent as defined in the section “Cell binding agent” above, the derivative must include a moiety (linking group) that allows the derivatives to be linked to a cell binding agent via a linkage such as a disulfide bond, a sulfide (or called herein thioether) bond, an acid-labile group, a photo-labile group, a peptidase-labile group, or an esterase-labile group. The derivatives are prepared so that they contain a moiety necessary to link the leptomycin derivative to a cell binding agent via, for example, a disulfide bond, a thioether bond, an acid-labile group, a photo-labile group, a peptidase-labile group, or an esterase-labile group. In order to further enhance solubility in aqueous solutions, the linking group can contain a polyethylene glycol spacer. In an embodiment, a sulfide or disulfide linkage is used because the reducing environment of the targeted cell results in cleavage of the sulfide or disulfide and release of the derivatives with an associated increase in cytotoxicity.

Compounds of the present invention may be prepared by a variety of synthetic routes. The reagents and starting materials are commercially available, or readily synthesized by well-known techniques by one of ordinary skill in the art. Methods for synthesizing leptomycin derivatives that may be used in the cytotoxic conjugates of the present invention, along with methods for conjugating said leptomycin derivatives to cell binding agents such as antibodies, are described in detail in in European Patent Application No. 06290948.6.

The cytotoxic agent used in the cytotoxic conjugates according to the present invention may also be CC-1065 or a derivative thereof.

CC-1065 is a potent anti-tumor antibiotic isolated from the culture broth of Streptomyces zelensis. CC-1065 is about 1000-fold more potent in vitro than are commonly used anti-cancer drugs, such as doxorubicin, methotrexate and vincristine (Bhuyan et al. (1982) Cancer Res. 42:3532-3537). CC-1065 and its analogs are disclosed in U.S. Pat. Nos. 6,372,738, 6,340,701, 5,846,545 and 5,585,499.

The cytotoxic potency of CC-1065 has been correlated with its alkylating activity and its DNA-binding or DNA-intercalating activity. These two activities reside in separate parts of the molecule. Thus, the alkylating activity is contained in the cyclopropapyrroloindole (CPI) subunit and the DNA-binding activity resides in the two pyrroloindole subunits.

Although CC-1065 has certain attractive features as a cytotoxic agent, it has limitations in therapeutic use. Administration of CC-1065 to mice caused a delayed hepatotoxicity leading to mortality on day 50 after a single intravenous dose of 12.5 μg/kg (Reynolds et al. (1986) J. Antibiotics XXIX:319-334). This has spurred efforts to develop analogs that do not cause delayed toxicity, and the synthesis of simpler analogs modeled on CC-1065 has been described (Warpehoski et al. (1988) J. Med. Chem. 31: 590-603).

In another series of analogs, the CPI moiety was replaced by a cyclopropabenzindole (CBI) moiety (Boger et al. (1990) J. Org. Chem. 55:5823-5833; Boger et al. (1991) Bio Org. Med. Chem. Lett. 1:115-120). These compounds maintain the high in vitro potency of the parental drug, without causing delayed toxicity in mice. Like CC-1065, these compounds are alkylating agents that bind to the minor groove of DNA in a covalent manner to cause cell death. However, clinical evaluation of the most promising analogs, Adozelesin and Carzelesin, has led to disappointing results (Foster et al. (1996) Investigational New Drugs 13:321-326; Wolff et al. (1996) Clin. Cancer Res. 2:1717-1723). These drugs display poor therapeutic effects because of their high systemic toxicity.

The therapeutic efficacy of CC-1065 analogs can be greatly improved by changing the in vivo distribution through targeted delivery to the tumor site, resulting in lower toxicity to non-targeted tissues, and thus, lower systemic toxicity. In order to achieve this goal, conjugates of analogs and derivatives of CC-1065 with cell-binding agents that specifically target tumor cells have been described (U.S. Pat. Nos. 5,475,092; 5,585,499; 5,846,545). These conjugates typically display high target-specific cytotoxicity in vitro, and exceptional anti-tumor activity in human tumor xenograft models in mice (Chari et al. (1995) Cancer Res. 55:4079-4084).

Recently, prodrugs of CC-1065 analogs with enhanced solubility in aqueous medium have been described (European Patent Application No. 06290379.4). In these prodrugs, the phenolic group of the alkylating portion of the molecule is protected with a functionality that renders the drug stable upon storage in acidic aqueous solution, and confers increased water solubility to the drug compared to an unprotected analog. The protecting group is readily cleaved in vivo at physiological pH to give the corresponding active drug. In the prodrugs described in EP 06290379.4, the phenolic substituent is protected as a sulfonic acid containing phenyl carbamate which possesses a charge at physiological pH, and thus has enhanced water solubility. In order to further enhance water solubility, an optional polyethylene glycol spacer can be introduced into the linker between the indolyl subunit and the cleavable linkage such as a disulfide group. The introduction of this spacer does not alter the potency of the drug.

Methods for synthesizing CC-1065 analogs that may be used in the cytotoxic conjugates of the present invention, along with methods for conjugating the analogs to cell binding agents such as antibodies, are described in detail in EP 06290379.4 and U.S. Pat. Nos. 5,475,092, 5,846,545, 5,585,499, 6,534,660 and 6,586,618 and in U.S. application Ser. Nos. 10/116,053 and 10/265,452.

Drugs such as methotrexate, daunorubicin, doxorubicin, vincristine, vinblastine, melphalan, mitomycin C, chlorambucil, calicheamicin, tubulysin and tubulysin analogs, duocarmycin and duocarmycin analogs, dolastatin and dolastatin analogs are also suitable for the preparation of conjugates of the present invention. The drug molecules can also be linked to the antibody molecules through an intermediary carrier molecule such as serum albumin. Doxarubicin and Danorubicin compounds, as described, for example, in U.S. Pat. No. 6,630,579, may also be useful cytotoxic agents.

In a particular embodiment of the invention, the at least one cytotoxic agent is the maytansine DM1 of formula (I). In another particular embodiment of the invention, the at least one cytotoxic agent is the maytansine DM4 of formula (II).

These cytotoxic agents are conjugated to the cell binding agents, antibodies, epitope-binding fragments of antibodies as disclosed herein.

### Linker

“Linker”, as used herein, means a chemical moiety comprising a covalent bond or a chain of atoms that covalently attaches a polypeptide to a drug moiety.

The conjugates may be prepared by in vitro methods. In order to link a drug or prodrug to the cell binding agent, in particular to the antibody, a linking group is used. Suitable linking groups are well known in the art and include disulfide groups, thioether groups, acid labile groups, photolabile groups, peptidase labile groups and esterase labile groups.

Conjugation of a cell binding agent as defined in the section “Cell binding agent” above, in particular an antibody of the invention, with cytotoxic agents as defined in the section “Cytotoxic agent” above may be made using a variety of bifunctional protein coupling agents including but not limited to N-succinimidyl pyridyldithiobutyrate (SPDB), butanoic acid 4-[(5-nitro-2-pyridinyl)dithio]-2,5-dioxo-1-pyrrolidinyl ester (nitro-SPDB), 4-(Pyridin-2-yldisulfanyl)-2-sulfo-butyric acid (sulfo-SPDB), N-succinimidyl (2-pyridyldithio) propionate (SPDP), succinimidyl (N-maleimidomethyl) cyclohexane-1-carboxylate (SMCC), iminothiolane (IT), bifunctional derivatives of imidoesters (such as dimethyl adipimidate HCL), active esters (such as disuccinimidyl suberate), aldehydes (such as glutaraldehyde), bis-azido compounds (such as bis-(p-azidobenzoyl)-hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene).

In a particular embodiment, said linker is selected from the group consisting of N-succinimidyl pyridyldithiobutyrate (SPDB), 4-(Pyridin-2-yldisulfanyl)-2-sulfo-butyric acid (sulfo-SPDB), and succinimidyl (N-maleimidomethyl) cyclohexane-1-carboxylate (SMCC).

The cell binding agent of the conjugate of the invention may be covalently linked via a cleavable or non-cleavable linker to the at least one cytotoxic agent.

The linker may be a “cleavable linker” facilitating release of the cytotoxic agent in the cell. For example, an acid-labile linker, a peptidase-sensitive linker, an esterase labile linker, a photolabile linker or a disulfide-containing linker (see e.g. U.S. Pat. No. 5,208,020) may be used. The linker may be also a “non-cleavable linker” (for example SMCC linker) that might lead to better tolerance in some cases.

Alternatively, a fusion protein comprising the cell binding agent as defined in the section “Cell binding agent” above, in particular the antibody, of the invention and a cytotoxic polypeptide may be made, by recombinant techniques or peptide synthesis. The length of DNA may comprise respective regions encoding the two portions of the conjugate either adjacent one another or separated by a region encoding a linker peptide which does not destroy the desired properties of the conjugate.

The cell binding agents, in particular the antibodies, of the present invention may also be used in Dependent Enzyme Mediated Prodrug Therapy by conjugating the polypeptide to a prodrug-activating enzyme which converts a prodrug (e.g. a peptidyl chemotherapeutic agent, see WO81/01145) to an active anti-cancer drug (see, for example, WO88/07378 and U.S. Pat. No. 4,975,278). The enzyme component of the immunoconjugate useful for ADEPT includes any enzyme capable of acting on a prodrug in such a way so as to convert it into its more active, cytotoxic form. Enzymes that are useful in the method of this invention include, but are not limited to, alkaline phosphatase useful for converting phosphate-containing prodrugs into free drugs; arylsulfatase useful for converting sulfate-containing prodrugs into free drugs; cytosine deaminase useful for converting non-toxic fluorocytosine into the anticancer drug, 5-fluorouracil; proteases, such as serratia protease, thermolysin, subtilisin, carboxypeptidases and cathepsins (such as cathepsins B and L), that are useful for converting peptide-containing prodrugs into free drugs; D-alanylcarboxypeptidases, useful for converting prodrugs that contain D-amino acid substituents; carbohydrate-cleaving enzymes such as O-galactosidase and neuraminidase useful for converting glycosylated prodrugs into free drugs; P-lactamase useful for converting drugs derivatized with P-lactams into free drugs; and penicillin amidases, such as penicillin V amidase or penicillin G amidase, useful for converting drugs derivatized at their amine nitrogens with phenoxyacetyl or phenylacetyl groups, respectively, into free drugs. The enzymes can be covalently bound to the polypeptides of the invention by techniques well known in the art such as the use of the heterobifunctional crosslinking reagents discussed above.

According to a particular embodiment, in the conjugate of the invention, the cytotoxic agent may be a maytansinoid, in particular DM1 or DM4.

In such a conjugate, the cell binding agent as defined in the section “Cell binding agent” above, in particular the antibody, is conjugated to said at least one cytotoxic agent by a linking group. In particular said linking group is a non-cleavable linker, such as SPDB, sulfo-SPDB, or SMCC.

In a particular embodiment, said linker is N-succinimidyl pyridyldithiobutyrate (SPDB) and said cytotoxic agent is DM4. In another particular embodiment, said linker is 4-(Pyridin-2-yldisulfanyl)-2-sulfo-butyric acid (sulfo-SPDB) and said cytotoxic agent is DM4.

More particularly, the conjugate may be selected from the group consisting of:

i) an antibody-SPDB-DM4 conjugate of formula (XVIII)

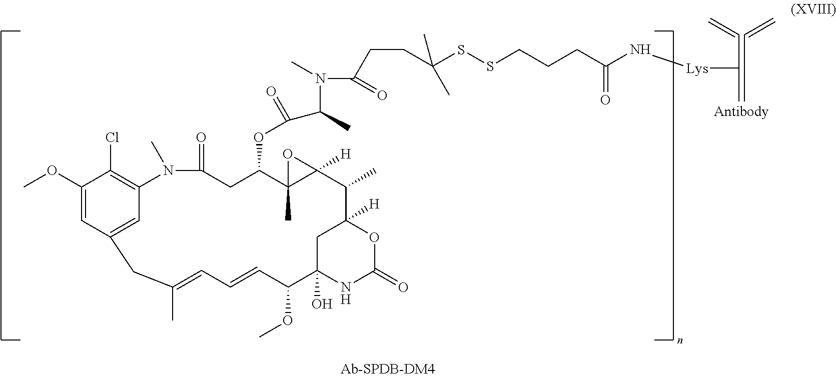

ii) an antibody-sulfo-SPDB-DM4 conjugate of formula (XIX)

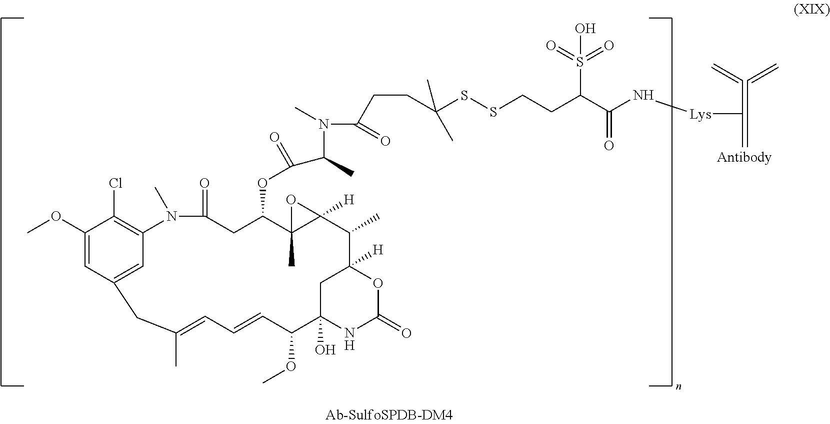

iii) an antibody-SMCC-DM1 conjugate of formula (XX)

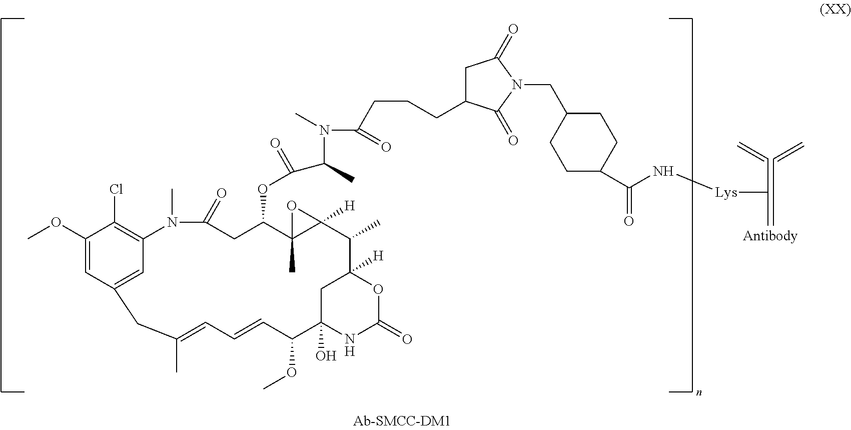

In general, the conjugate can be obtained by a process comprising the steps of:

(i) bringing into contact an optionally-buffered aqueous solution of a cell-binding agent (e.g. an antibody according to the invention) with solutions of a linker and a cytotoxic compound;
(ii) then optionally separating the conjugate which was formed in (i) from the unreacted cell-binding agent.

The aqueous solution of cell-binding agent can be buffered with buffers such as, e.g. potassium phosphate, acetate, citrate or N-2-Hydroxyethylpiperazine-N′-2-ethanesulfonic acid (Hepes buffer). The buffer depends upon the nature of the cell-binding agent. The cytotoxic compound is in solution in an organic polar solvent, e.g. dimethyl sulfoxide (DMSO) or dimethylacetamide (DMA).

The reaction temperature is usually comprised between 20° C. and 40° C. The reaction time can vary from 1 to 24 h. The reaction between the cell-binding agent and the cytotoxic agent can be monitored by size exclusion chromatography (SEC) with a refractometric and/or UV detector. If the conjugate yield is too low, the reaction time can be extended.

A number of different chromatography methods can be used by the person skilled in the art in order to perform the separation of step (ii): the conjugate can be purified e.g. by SEC, adsorption chromatography (such as ion exchange chromatography, IEC), hydrophobic interaction chromatograhy (HIC), affinity chromatography, mixed-support chromatography such as hydroxyapatite chromatography, or high performance liquid chromatography (HPLC). Purification by dialysis or diafiltration can also be used.

As used herein, the term “aggregates” means the associations which can be formed between two or more cell-binding agents, said agents being modified or not by conjugation. The aggregates can be formed under the influence of a great number of parameters, such as a high concentration of cell-binding agent in the solution, the pH of the solution, high shearing forces, the number of bonded dimers and their hydrophobic character, the temperature (see Wang and Gosh (2008) J. Membr Sci. 318: 311-316, and references cited therein); note that the relative influence of some of these parameters is not clearly established. In the case of proteins and antibodies, the person skilled in the art will refer to Cromwell et al. (2006) AAPS Jounal 8:E572-E579. The content in aggregates can be determined with techniques well known to the skilled person, such as SEC (see Walter et al. (1993) Anal. Biochem. 212:469-480.

After step (i) or (ii), the conjugate-containing solution can be submitted to an additional step (iii) of chromatography, ultrafiltration and/or diafiltration.

The conjugate is recovered at the end of these steps in an aqueous solution.

In the embodiments of the invention wherein the cytotoxic agent is a maytansinoid, in order to link the maytansinoid to the cell binding agent as defined in the section “Cell binding agent” above, such as the humanized huDS6 antibody comprising a heavy chain of sequence SEQ ID NO: 9 and a light chain of sequence SEQ ID NO: 10, the maytansinoid may comprise a linking moiety. The linking moiety contains a chemical bond that allows for the release of fully active maytansinoids at a particular site. Suitable chemical bonds are well known in the art and include disulfide bonds, acid labile bonds, photolabile bonds, peptidase labile bonds and esterase labile bonds. Preferred are disulfide bonds.

The linking moiety also comprises a reactive chemical group. In an embodiment, the reactive chemical group can be covalently bound to the maytansinoid via a disulfide bond linking moiety.

Particular reactive chemical groups are N-succinimidyl esters and N-sulfosuccinimidyl esters.

Particular maytansinoids comprising a linking moiety that contains a reactive chemical group are C-3 esters of maytansinol and its analogs where the linking moiety contains a disulfide bond and the chemical reactive group comprises a N-succinimidyl or N-sulfosuccinimidyl ester.

Many positions on maytansinoids can serve as the position to chemically link the linking moiety. For example, the C-3 position having a hydroxyl group, the C-14 position modified with hydroxymethyl, the C-15 position modified with hydroxy and the C-20 position having a hydroxy group are all expected to be useful. However the C-3 position is preferred and the C-3 position of maytansinol is especially preferred.

While the synthesis of esters of maytansinol having a linking moiety is described in terms of disulfide bond-containing linking moieties, one of skill in the art will understand that linking moieties with other chemical bonds (as described above) can also be used with the present invention, as can other maytansinoids. Specific examples of other chemical bonds include acid labile bonds, photolabile bonds, peptidase labile bonds and esterase labile bonds. The disclosure of U.S. Pat. No. 5,208,020 teaches the production of maytansinoids bearing such bonds.

The synthesis of maytansinoids and maytansinoid derivatives having a disulfide moiety that bears a reactive group is described in U.S. Pat. Nos. 6,441,163 and 6,333,410, and U.S. application Ser. No. 10/161,651.

The reactive group-containing maytansinoids, such as DM1, are reacted with a cell binding agent as defined in the section “Cell binding agent” above, in particular with an antibody, such as the humanized huDS6 antibody comprising a heavy chain of sequence SEQ ID NO: 9 and a light chain of sequence SEQ ID NO: 10, to produce cytotoxic conjugates. These conjugates may be purified by HPLC or by gel-filtration.

Several excellent schemes for producing such cell binding agent-maytansinoid, in particular antibody-maytansinoid conjugates are provided in U.S. Pat. No. 6,333,410, and U.S. application Ser. Nos. 09/867,598, 10/161,651 and 10/024,290.

In general, a solution of an antibody in aqueous buffer may be incubated with a molar excess of maytansinoids having a disulfide moiety that bears a reactive group. The reaction mixture can be quenched by addition of excess amine (such as ethanolamine, taurine, etc.). The maytansinoid-antibody conjugate may then be purified by gel-filtration.

The number of maytansinoid molecules bound per antibody molecule can be determined by measuring spectrophotometrically the ratio of the absorbance at 252 nm and 280 nm. An average of 1-10 maytansinoid molecules/antibody molecule is preferred.

Maytansinoids may also be linked to cell binding agents using PEG linking groups, as set forth in U.S. application Ser. No. 10/024,290. These PEG linking groups are soluble both in water and in non-aqueous solvents, and can be used to join one or more cytotoxic agents to a cell binding agent. Exemplary PEG linking groups include hetero-bifunctional PEG linkers that bind to cytotoxic agents and cell binding agents at opposite ends of the linkers through a functional sulfhydryl or disulfide group at one end, and an active ester at the other end.

As a general example of the synthesis of a cytotoxic conjugate using a PEG linking group, reference is again made to U.S. application Ser. No. 10/024,290 for specific details. Synthesis begins with the reaction of one or more cytotoxic agents bearing a reactive PEG moiety with a cell-binding agent, resulting in displacement of the terminal active ester of each reactive PEG moiety by an amino acid residue of the cell binding agent, such as the humanized huDS6 antibody comprising a heavy chain of sequence SEQ ID NO: 9 and a light chain of sequence SEQ ID NO: 10, to yield a cytotoxic conjugate comprising one or more cytotoxic agents covalently bonded to a cell binding agent through a PEG linking group.

The conjugate molecules of the invention may be formed using any techniques. In particular, the tomaymycin derivatives of the invention may be linked to an antibody or other cell binding agent as defined in the section “Cell binding agent” above via an acid labile linker, or by a photolabile linker. The derivatives can be condensed with a peptide having a suitable sequence and subsequently linked to a cell binding agent to produce a peptidase labile linker. The conjugates can be prepared to contain a primary hydroxyl group, which can be succinylated and linked to a cell binding agent to produce a conjugate that can be cleaved by intracellular esterases to liberate free derivative. Preferably, the derivatives are synthesized to contain a free or protected thiol group, and then one or more disulfide or thiol-containing derivatives are each covalently linked to the cell binding agent via a disulfide bond or a thioether link.

Numerous methods of conjugation are taught in U.S. Pat. Nos. 5,416,064 and 5,475,092. The tomaymycin derivatives can be modified to yield a free amino group and then linked to an antibody or other cell binding agent via an acid labile linker or a photolabile linker. The tomaymycin derivatives with a free amino or carboxyl group can be condensed with a peptide and subsequently linked to a cell binding agent to produce a peptidase labile linker. The tomaymycin derivatives with a free hydroxyl group on the linker can be succinylated and linked to a cell binding agent to produce a conjugate that can be cleaved by intracellular esterases to liberate free drug. Most preferably, the tomaymycin derivatives are treated to create a free or protected thiol group, and then the disulfide- or thiol containing tomaymycin dimers are linked to the cell binding agent via disulfide bonds.

In one embodiment, monoclonal antibody- or cell binding agent-tomaymycin derivative conjugates are those that are joined via a disulfide bond, as discussed above, that are capable of delivering tomaymycin derivatives. Such cell binding conjugates are prepared by known methods such as by modifying monoclonal antibodies with succinimidyl pyridyl-dithiopropionate (SPDP) (Carlsson et al. (1978) Biochem. J. 173:723-737). The resulting thiopyridyl group is then displaced by treatment with thiol-containing tomaymycin derivatives to produce disulfide linked conjugates. Alternatively, in the case of the aryldithio-tomaymycin derivatives, the formation of the cell binding conjugate is effected by direct displacement of the aryl-thiol of the tomaymycin derivative by sulfhydryl groups previously introduced into antibody molecules. Conjugates containing 1 to 10 tomaymycin derivative drugs linked via a disulfide bridge are readily prepared by either method.

More specifically, a solution of the dithio-nitropyridyl modified antibody at a concentration of 2.5 mg/ml in 0.05 M potassium phosphate buffer, at pH 7.5 containing 2 mM EDTA is treated with the thiol-containing tomaymycin derivative (1.3 molar eq./dithiopyridyl group). The release of thio-nitropyridine from the modified antibody is monitored spectrophotometrically at 325 nm and is complete in about 16 h. The antibody-tomaymycin derivative conjugate is purified and freed of unreacted drug and other low molecular weight material by gel filtration through a column of Sephadex G-25 or Sephacryl S300. The number of tomaymycin derivative moieties bound per antibody molecule can be determined by measuring the ratio of the absorbance at 230 nm and 275 nm. An average of 1-10 tomaymycin derivative molecules/antibody molecule can be linked via disulfide bonds by this method.

The effect of conjugation on binding affinity towards the antigen-expressing cells can be determined using the methods previously described by Liu et al. (1996) Proc. Natl. Acad. Sci. U.S.A. 93:8618-8623. Cytotoxicity of the tomaymycin derivatives and their antibody conjugates to cell lines can be measured by back-extrapolation of cell proliferation curves as described in Goldmacher et al. (1985) J. Immunol. 135:3648-3651. Cytotoxicity of these compounds to adherent cell lines can be determined by clonogenic assays as described in Goldmacher et al. (1986) J. Cell Biol. 102:1312-1319.

### Drug-to-Antibody Ratio

According to an embodiment, the conjugate according to the invention is characterised by a “drug-to-antibody ratio” (or “DAR”) as measured by DAR UV ranging from 1 to 10, for instance from 2 to 5, in particular from 3 to 4, more particularly of 3.5. This is generally the case of conjugates including maytansinoid molecules.

This DAR number can vary with the nature of the cell binding agent, in particular the antibody, and of the drug (i.e. the cytotoxic agent) used along with the experimental conditions used for the conjugation (like the ratio cytotoxic agent/cell binding agent, the reaction time, the nature of the solvent and of the cosolvent if any). Thus the contact between the cell binding agent and the cytotoxic agent leads to a mixture comprising several conjugates differing from one another by different drug-to-antibody ratios; optionally the naked cell binding agent; optionally aggregates. The DAR that is determined is thus a mean value.

A method which can be used to determine the DAR, herein called DAR UV, consists in measuring spectrophotometrically the ratio of the absorbance at of a solution of substantially purified conjugate at λ_{D} and 280 nm. 280 nm is a wavelength generally used for measuring protein concentration, such as antibody concentration. The wavelength λ_{D} is selected so as to allow discriminating the drug from the antibody, i.e. as readily known to the skilled person, λ_{D} is a wavelength at which the drug has a high absorbance and λ_{D} is sufficiently remote from 280 nm to avoid substantial overlap in the absorbance peaks of the drug and antibody. λ_{D} may be selected as being 252 nm in the case of maytansinoid molecules. A method of DAR calculation may be derived from Antony S. Dimitrov (ed), LLC, 2009, Therapeutic Antibodies and Protocols, vol 525, 445, Springer Science:

The absorbances for the conjugate at λ_{D} (A_{λD}) and at 280 nm (A₂₈₀) are measured using a classic spectrophotometer apparatus (allowing to calculate the “DAR parameter”). The absorbances can be expressed as follows:

A_{λD}=(c_{D}×ε_{DλD})+(c_{A}×ε_{AλD})

A₂₈₀=(c_{D}×ε_{D280})+(c_{A}×ε_{A280})

wherein:
c_{D} and c_{A} are respectively the concentrations in the solution of the drug and of the antibody
ε_{DλD} and ε_{D280} are respectively the molar extinction coefficients of the drug at λ_{D} and 280 nm
ε_{AλD} and ε_{A280} are respectively the molar extinction coefficients of the antibody at λ_{D} and 280 nm.

Resolution of these two equations with two unknowns leads to the following equations:

c_{D}=[(ε_{A280}×A_{λD})−(εAλD×A₂₈₀)]/[(ε_{DλD}×ε_{A280})−(ε_{AλD}×ε_{D280})]

c_{A}=[A₂₈₀−(c_{D}×ε_{D280})]/ε_{A280}

The average DAR is then calculated from the ratio of the drug concentration to that of the antibody: DAR=c_{D}/c_{A}.

In a particular embodiment, λ_{D} is 252 nm.

Accordingly, in that particular embodiment, the conjugate is characterized by a drug-to-antibody ratio (DAR) ranging from 3 to 4, in particular of 3.5, the DAR being calculated from the ratio of the cytotoxic agent concentration (c_{D}) to that of the cell binding agent (c_{A});

$$DAR = \frac{{c}_{D}}{{c}_{A}}$$

$$wherein$$

$${c}_{D} = [ ( {ɛ}_{A 280} \times {A}_{252} ) - ( {ɛ}_{A 252} \times {A}_{280} ) ] / [ ( {ɛ}_{D 252} \times {ɛ}_{A 280} ) - ( {ɛ}_{A 252} \times {ɛ}_{D 280} ) ]$$

$${c}_{A} = [ {A}_{280} - ( {c}_{D} \times {ɛ}_{D 280} ) ] / {ɛ}_{A 280}$$

and
ε_{D252} and εD₂₈₀ are respectively the molar extinction coefficients of the cytotoxic agent at 252 nm and 280 nm,
ε_{A252} and ε_{A280} are respectively the molar extinction coefficients of the cell binding agent at 252 nm and 280 nm, and
A₂₅₂ and A₂₈₀ are respectively the absorbances for the conjugate at 252 nm (A₂₅₂) and at 280 nm (A₂₈₀), measured using a classic spectrophotometer apparatus.

### Treatment

The inventors demonstrated that a patient suffering from cancer, in particular from breast cancer or ovarian cancer, more particularly of breast cancer, showed at least a particular response when she was administrated with a dose of at least 120 mg/m² of the conjugate SAR566658.

The present invention thus concerns a conjugate comprising (i) a cell binding agent which binds to the human mucin-1 (MUC1) glycoprotein, as defined in the section “Cell binding agent” herein above, linked to (ii) at least one cytotoxic agent, as defined in the section “Cytotoxic agent” herein above, for use to treat cancer, wherein said conjugate is administered at a dose of at least 120 mg/m².

The present invention also concerns the use of a conjugate comprising (i) a cell binding agent which binds to the human mucin-1 (MUC1) glycoprotein, as defined in the section “Cell binding agent” herein above, linked to (ii) at least one cytotoxic agent, as defined in the section “Cytotoxic agent” herein above, for the manufacture of a medicament intended to treat cancer, wherein said conjugate is administered at a dose of at least 120 mg/m².

The present invention also concerns a method for treating cancer in a patient comprising administering to a patient in need thereof a conjugate comprising (i) a cell binding agent which binds to the human mucin-1 (MUC1) glycoprotein, as defined in the section “Cell binding agent” herein above, linked to (ii) at least one cytotoxic agent, as defined in the section “Cytotoxic agent” herein above at a dose of at least 120 mg/m².

In the context of the invention, the term “treating” or “treatment”, as used herein, means reversing, alleviating, inhibiting the progress of, or preventing the disorder or condition to which such term applies, or one or more symptoms of such disorder or condition.

By the term “treating cancer” as used herein is meant the inhibition of the growth of malignant cells of a tumour and/or the progression of metastases from said tumor. Such treatment can also lead to the regression of tumor growth, i.e., the decrease in size of a measurable tumor. In a particular embodiment, such treatment leads to a partial regression of the tumor or metastase. In another particular embodiment, such treatment leads to the complete regression of the tumor or metastase.

According to the invention, the term “patient” or “patient in need thereof” is intended for a human or non-human mammal affected or likely to be affected with a malignant tumor.

In a particular embodiment, the patient to be treated may have been previously treated with other anti-cancer treatments. In particular, the patient to be treated may have been previously treated with an oxaliplatin-, cisplatin-, a carboplatin-, and/or a paclitaxel-docetaxel-based regimen.

By a “therapeutically effective amount” of the conjugate of the invention is meant a sufficient amount of the conjugate to treat said cancer disease, at a reasonable benefit/risk ratio applicable to any medical treatment. It will be understood, however, that the total daily usage of the conjugate of the present invention will be decided by the attending physician within the scope of sound medical judgment. The specific therapeutically effective dose level for any particular patient will depend upon a variety of factors including the disorder being treated and the severity of the disorder; activity of the specific conjugate employed; the specific composition employed, the age, body weight, general health, sex and diet of the patient; the time of administration, route of administration, and rate of excretion of the specific conjugate employed; the duration of the treatment; drugs used in combination or coincidental with the specific conjugate employed; and like factors well known in the medical arts.

In a particular embodiment, said therapeutically effective amount of the conjugate administered to the patient is a dose ranging from 120 mg/m² to 240 mg/m², more particularly ranging from 150 mg/m² to 240 mg/m², in particular a dose of 190 mg/m².

In a further embodiment, the conjugate of the invention is administered repeatedly according to a protocol that depends on the patient to be treated (age, weight, treatment history, etc.), which can be determined by a skilled physician. In one aspect of the invention, the conjugate of the invention is administered to the patient according to an intermittent program with an interval between each administration of 3 weeks, which may be prolonged by 1 to 2 weeks depending on the tolerance to the preceding administration. Accordingly, in a particular embodiment, the administration of the conjugate is repeated as a new cycle every 3 weeks.

In a further embodiment, the median number of cycles is of 2.

The conjugate of the invention may be administered in the form of a pharmaceutical composition including pharmaceutically acceptable excipients, and optionally sustained-release matrices, such as biodegradable polymers, to form therapeutic compositions.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The form of the pharmaceutical compositions including the conjugate of the invention and the route of administration naturally depend upon the condition to be treated, the severity of the illness, the age, weight, and gender of the patient, etc.

The conjugates of the invention can be formulated for a topical, oral, parenteral, intranasal, intravenous, intramuscular, subcutaneous or intraocular administration and the like. In a particular embodiment, the conjugate of the invention is administered intravenously

In particular, the pharmaceutical compositions including the conjugate of the invention may contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

To prepare pharmaceutical compositions, an effective amount of the conjugate of the invention may be dissolved or dispersed in a pharmaceutically acceptable carrier or aqueous medium.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

The carrier can be a solvent or dispersion medium containing, for example, water, ethanol, polyol (for example, glycerol, propylene glycol, and liquid polyethylene glycol, and the like) and suitable mixtures thereof. The proper fluidity can be maintained, for example, by the use of a coating, such as lecithin, by the maintenance of the required particle size in the case of dispersion and by the use of surfactants, stabilizing agents, cryoprotectants or antioxidants. The prevention of the action of microorganisms can be brought about by antibacterial and antifungal agents. In many cases, it will be preferable to include isotonic agents, for example, sugars or sodium chloride.

Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

In a particular embodiment, the conjugate of the invention is suitably administered intravenously at a rate of 1 mL/min for 30 min and then increased to a maximal rate of 2 mL/min in the absence of hypersensitivity reactions.

Cancers to be treated according to the invention include malignancy of any type, in particular solid tumors, for example breast cancer and ovarian cancer.

In one embodiment, the cancer to be treated according to the invention is a CA6-positive tumor. In a further embodiment, the cancer to be treated is a breast cancer, more particularly a triple negative breast cancer, not positive to receptors for estrogen, progesterone or HER2.

The conjugate of the invention may be administered in combination with a medication to prevent or control keratitis, in particular with a keratitis prophylactic or curative ocular composition.

### BRIEF DESCRIPTION OF THE SEQUENCES

| SEQ | | |
|---|---|---|
| ID | Sequence | Description |
| 1 | SYNMH | CDR1-H of huDS6 |
| 2 | YIYPGNGATNYNQKFKG | CDR2-H of huDS6 |
| 3 | GDSVPFAY | CDR3-H of huDS6 |
| 4 | SAHSSVSFMH | CDR1-L of huDS6 |
| 5 | STSSLAS | CDR2-L of huDS6 |
| 6 | QQRSSFPLT | CDR3-L of huDS6 |
| 7 | QAQLVQSGAEVVKPGASVKMSCKASGYTFTSYN | Heavy chain variable |
| | MHWVKQTPGQGLEWIGYIYP | region of huDS6 |
| | GNGATNYNQKFQGKATLTADPSSSTAYMQISSLTS | |
| | EDSAVYFCARGDSVPFAYW GQGTLVTVSA | |
| 8 | EIVLTQSPATMSASPGERVTITCSAHSSVSFMHWF | Light chain variable |
| | QQKPGTSPKLWIYSTSSLAS | region of huDS6 |
| | GVPARFGGSGSGTSYSLTISSMEAEDAATYYCQQ | |
| | RSSFPLTFGAGTKLELKR | |
| 9 | QAQLVQSGAEVVKPGASVKMSCKASGYTFTSYN | Heavy chain of huDS6 |
| | MHWVKQTPGQGLEWIGYIYPGNGATNYNQKFQG | |
| | KATLTADPSSSTAYMQISSLTSEDSAVYFCARGDS | |
| | VPFAYWGQGTLVTVSAASTKGPSVFPLAPSSKST | |
| | SGGTAALGCLVKDYFPEPVTVSWNSGALTSGVHT | |
| | FPAVLQSSGLYSLSSVVTVPSSSLGTQTYICNVNH | |
| | KPSNTKVDKKVEPKSCDKTHTCPPCPAPELLGGP | |
| | SVFLFPPKPKDTLMISRTPEVTCVVVDVSHEDPEV | |
| | KFNVVYVDGVEVHNAKTKPREEQYNSTYRVVSVLT | |
| | VLHQDWLNGKEYKCKVSNKALPAPIEKTISKAKGQ | |
| | PREPQVYTLPPSRDELTKNQVSLTCLVKGFYPSDI | |
| | AVEWESNGQPENNYKTTPPVLDSDGSFFLYSKLT | |
| | VDKSRWQQGNVFSCSVMHEALHNHYTQKSLSLS | |
| | PGK | |
| 10 | EIVLTQSPATMSASPGERVTITCSAHSSVSFMHWF | Heavy chain of huDS6 |
| | QQKPGTSPKLWIYSTSSLASGVPARFGGSGSGTS | |
| | YSLTISSMEAEDAATYYCQQRSSFPLTFGAGTKLE | |
| | LKRTVAAPSVFIFPPSDEQLKSGTASVVCLLNNFYP | |
| | REAKVQWKVDNALQSGNSQESVTEQDSKDSTYS | |
| | LSSTLTLSKADYEKHKVYACEVTHQGLSSPVTKSF | |
| | NRGEC | |

### BRIEF DESCRIPTION OF THE FIGURES

FIG. 1 summarizes patients treated by dose level and key events taken into account in the dose escalation determination.

FIG. 2 shows the worst grade ocular toxicity observed during the treatment displayed in the example (per patient and per cycle).

FIG. 3 shows DLCO decrease (per patient and per cycle) measured in section 2.6.3. of the example.

### EXAMPLE

### Materials and Methods

### Trial Design

This trial was designed as an open-label, dose-escalation study of the compound SAR566658 administered as a single agent by intravenous (IV) infusion, every 3 weeks, in adult patients with CA6-positive and refractory solid tumors to determine the maximal tolerated dose (MTD) of SAR566658.

### Primary Endpoint

To determine dose-limiting toxicity (DLT) and Maximum Tolerated dose (MTD) of SAR566658 IV every 3 weeks, toxicities were graded according to the National Cancer Institute Common Terminology Criteria for Adverse Events Version 4.03 (NCI CTCAE v.4.03).

Dose-limiting toxicity was defined as any of the following events unless unrelated to the SAR566658 compound during the first 3 weeks of study treatment:
Hematologic toxicity:
   Grade 4 neutropenia for 7 or more consecutive days,
   Febrile neutropenia or neutropenic infection,
   Grade 4 thrombocytopenia, or bleeding requiring transfusion with Grade 3 thrombocytopenia.
Non-hematologic toxicity:
   Grade 4 infusion reaction or Grade 3 infusion reaction if the infusion reaction did not resolve within 24 hours and the entire dose of IMP couldn't be administered,
   Grade 4 vomiting or Grade 3 nausea or vomiting not resolved to Grade ≦1 within 48 hours despite adequate antiemetic treatment,
   Any other Grade 3 or higher non-hematological clinical adverse event (AE),
   Any Grade 3 or higher laboratory abnormalities,
   Any toxicity related to SAR566658 resulting in a treatment delay of more than 2 weeks due to delayed recovery to baseline or Grade ≦1.

### Dose Escalation Rules

The 10 mg/m² dose was the starting dose level (DL) of SAR566658.

An accelerated dose escalation scheme was used for the two first DLs 10 mg/m² and 20 mg/m², based on toxicities observed during the first cycle of treatment: 1 patient per DL and 100% dose escalation between 2 DLs until the report of any Grade ≧2 SAR566658-related AE. If a SAR566658-related AE Grade ≧2 was reported by a patient, two additional patients were to be treated at the same DL and the dose escalation had to proceed with a classical scheme.

Even in the absence of toxicities, from DL of 40 mg/m², the dose escalation proceeded with a classical scheme (“3+3”). Doses were increased by 25% to 50%, instead of 100% and sequential cohorts of 3-6 patients each with CA6-positive advanced solid tumors have been treated with successively higher doses of SAR566658 every 3 weeks. Enrolment at the higher dose levels might not proceed until at least 3 patients treated at the current dose level have been followed for at least 3 weeks and the dose escalation criteria described below were met:

| Number of Patients | |
|---|---|
| with a DLT at | |
| Cycle 1 at a | |
| Given DL | Dose Escalation Decision Rule |
| 0 of first 3 | Enter at least 3 patients at the next dose level. |
| ≧2 out of 3 | Dose escalation will be stopped. Three (3) additional |
| | patients were entered at the previous DL if ≦3 |
| | patients were treated at that dose. |
| 1 out of 3 | Enter up to 6 patients at this DL. |
| | If 0 of the 3 additional patients experience DLT, then |
| | proceed to the next dose level. |
| | If 1 or more of up to 3 additional patients experience |
| | DLT, then dose escalation was stopped. Three (3) |
| | additional patients were entered at the previous DL if |
| | ≦3 patients were treated at that dose. |

### Study Population

Patient with a CA6-positive solid tumors for which no standard therapy was available.

The positivity of CA6, defined by immunohistochemistry (IHC) (i.e. moderate to intense membrane staining of ≧ than 30% of tumor cells) was assessed at a central laboratory on the most recent available tumor sample.

### SAR566558

Formulation: SAR566558 was supplied as a 25 mL extractable concentrate for solution for infusion of 125 mg contained in a 30 mL glass vial.

Route of administration: SAR566658 was administered by IV infusion at a rate of 1 mL/min for 30 minutes and then increased to a maximal rate of 2 mL/min in the absence of hypersensitivity reactions.

Dose regimen/duration: SAR566658 was administered on Day 1, repeated every 21 days. This constitutes one cycle of treatment. The patients might continue treatment until disease progression, unacceptable toxicity, or willingness to stop, followed by a minimum of 30-day visit.

The first trial cut-off date was planned 6 weeks after the last patient treated in the dose escalation phase (end of cycle 2) in order to have at least 2 evaluable cycles for all patients. The first trial cut-off date was actually performed 5 weeks after the last patient treated in the dose escalation phase. Therefore only cycle 1 of this patient was included.

### Study Period

Date of first patient treated: Sep. 15, 2010
Date of last patient treated: Jun. 12, 2013

### Number of Patients

Enrolled: 43

Treated: 34

Evaluable for:
Safety: 34
DLT: 34
Pharmacokinetic: 33
Pharmacodynamic: 34
Efficacy: 33

### Results

### 1. Study Patients

### 1.1. Patients Accountability

From Sep. 15, 2010 to Jun. 12, 2013, in 2 US sites and 2 Europe sites (1 in Spain and 1 in France), 43 patients entered the escalation step of this phase I study and 34 were treated.

### 1.2. Study Disposition

From the 34 patients, 28 discontinued study treatment, 6 are still under treatment. The most common reason for treatment discontinuation in the SAR566658 treated population was ‘progressive disease’ as described in Table 1. Other reason for treatment discontinuation was adverse events (AE) for 3 patients: #840 002 019 at 120 mg/m² (liver function tests increase in a pancreas cancer patient), #840 002 029 at 190 g/m² (non related pulmonary embolism in a pancreas cancer patient), #840 001 037 at 240 mg/m² (related diarrhea and vomiting).

TABLE 1
Reasons for study treatment discontinuation (treated population)

| | Initial planned SAR566658 dose level (mg/m²) | | | | | | |
|---|---|---|---|---|---|---|---|
| | ≦60 | 90 | 120 | 150 | 190 | 240 | All |
| Reasons^{a} | (N = 11) | (N = 3) | (N = 3) | (N = 3) | (N = 6) | (N = 8) | doses |
| Adverse experience | 0 | 0 | 1 | 0 | 1 | 1 | 3 |
| Progressive | 11 | 3 | 2 | 3 | 4 | 2 | 25 |
| disease^{b} | | | | | | | |
| Consent withdrawn | | | | | | | 0 |
| Others | | | | | | | 0 |
| All | 11/11 | 3/3 | 3/3 | 3/3 | 5/6 | 3/8 | 28/34 |

^{a}One reason per patient
^{b}Include radiologically documented disease progression and clinical and/or biological progression”

### 1.3. Demographics and Baseline Characteristics

Known patients' characteristics data at baseline are presented in Table 2.

The majority of patients are female (23/34, 68%), aged from 32 to 77 years (11 patients are ≧65) and had a good ECOG performance status (100% grade 0 or 1).

The primary tumor location was various, however ovarian cancer was the most frequent tumor (13/34, 38%), then pancreas (10/34, 29%) and breast (4/34, 12%). Carcinoma was the most frequent histological type, mainly adenocarcinoma (13/34, 38%) and epithelial cancer (13/34, 38%, all ovarian cancers).

The most frequent organs involved were: liver (18/34, 53%) peritoneum (13/34, 38%), lymph nodes (13/34, 38%), and lung (11/34, 32%).

TABLE 2
Demographics and Baseline Characteristics

| | Initial planned SAR566658 dose level (mg/m²) | | | | | | All |
|---|---|---|---|---|---|---|---|
| | ≦60 | 90 | 120 | 150 | 190 | 240 | doses |
| Total number of patients | 11 | 3 | 3 | 3 | 6 | 8 | 34 |
| Sex | | | | | | | |
| Male | 2 | 3 | 1 | 1 | 3 | 1 | 11 |
| | | | | | | | (32.4%) |
| Female | 9 | 0 | 2 | 2 | 3 | 7 | 23 |
| | | | | | | | (67.6%) |
| Age (years) | | | | | | | |
| Median (Min-Max) | 66 | 49 | 64 | 60 | 50 | 57 | 58.5 |
| | (32-70) | (37-64) | (55-65) | (58-63) | (48-77) | (42-70) | (32-77) |
| ≧65 | 6 | 0 | 1 | 0 | 2 | 2 | 11 |
| ECOG Performance | | | | | | | |
| Status before first | | | | | | | |
| infusion | | | | | | | |
| 0 | 5 | 1 | 2 | 2 | 2 | 5 | 17 |
| | | | | | | | (50%) |
| 1 | 6 | 2 | 1 | 1 | 4 | 3 | 17 |
| | | | | | | | (50%) |
| Anatomic Site of | | | | | | | |
| primary tumor | | | | | | | |
| Ovary | 4 | 0 | 2 | 0 | 1 | 6 | 13 |
| Pancreas | 4 | 1 | 1 | 0 | 3 | 1 | 10 |
| Breast | 2 | 0 | 0 | 1 | 1 | 0 | 4 |
| Head and Neck | 1 | 2 | 0 | 0 | 0 | 0 | 3 |
| Lung | 0 | 0 | 0 | 0 | 1 | 1 | 2 |
| Others^{a} | 0 | 0 | 0 | 2 | 0 | 0 | 2 |
| Number of organs | | | | | | | |
| involved | | | | | | | |
| Median (Min-Max) | 3 | 1 | 2 | 2 | 2.5 | 2.5 | 2.5 |
| | (1-4) | (1-4) | (1-3) | (1-4) | (2-3) | (1-4) | (1-4) |
| Main organs involved | | | | | | | |
| Liver | 6 | 2 | 0 | 2 | 5 | 3 | 18 |
| Peritoneum | 4 | 0 | 2 | 1 | 2 | 4 | 13 |
| Lung | 4 | 1 | 1 | 1 | 0 | 4 | 11 |
| Lymph nodes | 4 | 1 | 1 | 1 | 2 | 4 | 13 |
| Prior Radiation Therapy | | | | | | | |
| Yes | 5 | 2 | 1 | 2 | 3 | 0 | 13 |

^{a}Included bladder and endometrium cancers (1 patient each)

All patients were evaluable for CA6 expression (IHC) at study entry as described in Table 3. Twenty seven patients (27/34, 79.4%) had a CA6 positive tumor with at least 30% of positive tumor cells with 2+ and 3+ membrane staining intensity. Seven patients had a percentage of staining cells below this threshold as most of them were enrolled before the implementation of this threshold in amendment 3.

TABLE 3
Membrane CA6 expression (Immunohistochemistry assay)

| % staining cells at intensity score 2+ or 3+ by class | N (%) |
|---|---|
| [0-10[ | 6 (17.6%) |
| [10-20[ | 0 |
| [20-30[ | 1 |
| [30-50[ | 12 (35.3%) |
| [50-80[ | 12 (35.3%) |
| [80-100[ | 3 (8.8%) |

### 2. Results—Safety

### 2.1. Dosage and Duration

A total of 114 cycles were administered in 34 patients: 19 cycles at dose levels ≦60 mg/m², 7 cycles at dose level 90 mg/m², 17 cycles at dose level 120 mg/m², 23 cycles at dose level 150 mg/m², 18 cycles at dose level 190 mg/m² and 30 cycles at dose level 240 mg/m², as presented in Table 4.

Overall the median number of cycle is 2, ranged from 1 to 14 (at 120 mg/m²). However, the number of cycles received was higher at doses ≧120 mg/m² compared to lower doses where most of the patients discontinued due to disease progression after 1 or 2 cycles.

Few cycles delays (18/114 cycles) were observed and most of them were due to keratitis at doses ≧150 mg/m², which is an expected SAR566658 toxicity.

Very few SAR566658 doses were reduced (7/114 cycles) and 5 of them were reduced from 240 to 190 mg/m² due to keratitis (Table 4)

The relative dose intensity (RDI) is closed to 1 at all dose levels except at 240 mg/m² (0.79) due to cycle delay and/or dose reduction in 6/8 patients.

TABLE 4
Number of cycles - Dose modifications

| | Initial planned SAR566658 dose level (mg/m²) | | | | | | All |
|---|---|---|---|---|---|---|---|
| | ≦60 | 90 | 120 | 150 | 190 | 240 | doses |
| N of patients | 11 | 3 | 3 | 3 | 6 | 8 | 34 |
| N of patients with ≧1 | 1 | 0 | 2 | 3 | 2 | 5 | 13 |
| cycle delayed | | | | | | | |
| N of cycles | | | | | | | |
| Total | 19 | 7 | 17 | 23 | 18+ | 30+ | 114+ |
| Median | 2 | 2 | 2 | 8 | 2+ | 3.5 | 2 |
| [range] | [1-4] | [2-3] | [1-14] | [5-10] | [2-6] | [1-9] | [1-14] |
| N of Cycles delayed | 1 | 0 | 2 | 7 | 3 | 5 | 18 |
| Median RDI | 0.98 | 0.99 | 0.95 | 0.88 | 1.00 | 0.79 | |
| [range] | [0.9-1.0] | [1.0-1.0] | [0.8-1.0] | [0.8-0.9] | [0.8-1.0] | [0.7-1.0] | |
| Median Actual dose | 13.51 | 29.85 | 37.91 | 44.03 | 63.24 | 63.51 | — |
| intensity (mg/m²/week) | | | | | | | |

N Number,
RDI Relative Dose Intensity

### 2.2. Adverse Events

Treatment emergent AEs (TEAEs) were defined as AEs observed during the on-treatment period, defined as the period from the first dose to 30 days after the last dose of SAR566658.

Thirty-three (33/34, 97.1%) patients had at least one clinical TEAE all grades, regardless of relationship to study treatment (laboratory abnormalities are not reported here). No AE dose-dependent were observed except ocular events which were mainly observed from 150 mg/m² DL.

The most frequent clinical TEAE (all grades, regardless of relationship to study treatment, in at least 6 patients) were:
Asthenia/fatigue (HLT) (28 patients, 82.3%, including 16 patients with study-drug related event)
Decrease appetite (13 patients, 38.2%, including 4 patients with study-drug related event)
Keratitis (11 patients, 32.4%, all considered study-drug related event),
Gastrointestinal and abdominal pains (HLT) (10 patients, 29.4%),
Nausea (10 patients, 29.4%, including 6 patients with study-drug related event)
Peripheral neuropathy (HLT) (10 patients, 29.4%, including 5 patients with study-drug related event). Of note 3 patients had paresthesia or dysesthesia including 1 who had both paresthesia/dysesthesia and peripheral neuropathy at the same cycle. A total of 12 patients had a peripheral neurological event.
Dry eye (8 patients, 23.5%, including 5 patients with study-drug related event)
Constipation, vomiting, musculoskeletal and connective tissue pain (HLT) (each of events: 8 patients, 23.5%)
Diarrhea (7 patients, 20.6%)
Anxiety, Edema (HLT) (each of events: 6 patients, 17.6%).

Ocular event such as keratitis, dry eye as well as peripheral neuropathy are expected events with SAR566658, and are to be attributed to DM4-loaded ADC (see Section 2.6.1).

Overall TEAEs considered related to study treatment were by decreasing order: asthenia/fatigue (16 patients), keratitis, (11 patients), nausea, vomiting (6 patients each), peripheral neuropathy or paresthesia/dysesthesia (5 and 3 patients respectively), dry eye (5 patients), decrease appetite and blurred vision (4 patients each).

Eleven (32.3%) patients had at least one grade 3-4 TEAE (regardless on relationship to study treatment, excluding laboratory abnormalities): 1 at the each of the following dose level: 60, 90, 120 and 150 mg/m², 3 (50%) at the 190 mg/m² dose level, and 4 (50%) at the 240 mg/m² dose level. A total of four patients had at least one grade 3-4 clinical TEAE considered related to study treatment: keratitis (2 patients, including one patient with grade 3 blurred vision), vomiting and diarrhea (1 patient), and 1 patient with the following events: FEV1 decrease and ejection fraction decrease (in context of pulmonary embolism and disease progression). All but one were observed at 240 mg/m² DL. Two other patients had grade 3-4 laboratory abnormalities considered related to study treatment: neutropenia at 150 mg/m² (1 patient) and transaminases increase at 120 mg/m² (1 patient).

Hematological tests abnormalities (neutropenia, anemia and thrombocytopenia) were determined by blood evaluations collected on study treatment (Table 5). Two grade 3 neutropenia was observed at 150 and 190 mg/m² DLs, one lead to cycle delay.

TABLE 5
Hematological toxicity - Worst grade by patient

| | Initial planned SAR566658 dose level | | | | | | |
|---|---|---|---|---|---|---|---|
| | (mg/m²) | | | | | | All |
| | ≦60 | 90 | 120 | 150 | 190 | 240 | doses |
| Total number of treated patients | 11 | 3 | 3 | 3 | 6 | 8 | 34 |
| [N] | | | | | | | |
| Total number of evaluable | 11 | 3 | 3 | 3 | 6 | 8 | 34 |
| patients* [N] | | | | | | | |
| Leucopenia N (Gr 3-4 N) | 5 (1) | 1 (0) | 1 (0) | 2 (0) | 2 (0) | 4 (0) | 15 (0) |
| Total number of evaluable | 11 | 3 | 3 | 3 | 6 | 8 | 34 |
| patients* [N] | | | | | | | |
| Neutropenia N (Gr 3-4 N) | 2 (0) | 0 (0) | 0 (0) | 1 (1) | 1 (1) | 1 (0) | 5 (2) |
| Total number of evaluable | 11 | 3 | 3 | 3 | 6 | 8 | 34 |
| patients* [N] | | | | | | | |
| Anemia N (Gr 3-4 N) | 10 (0) | 3 (0) | 3 (0) | 2 (0) | 5 (0) | 7 (0) | 30 (0) |
| Total number of evaluable | 11 | 3 | 3 | 3 | 6 | 8 | 34 |
| patients* [N] | | | | | | | |
| Thrombocytopenia N (Gr 3-4 N) | 3 (0) | 0 (0) | 1 (0) | 1 (0) | 3 (0) | 2 (0) | 10 (0) |

*a patient is evaluable if having at least a blood count for the given test between two infusions.

Five pancreas cancer patients had severe liver function test abnormalities (2 patients with grade 3 transaminases AST or ALT, 4 patients with grade 3 alkaline phosphatase increase, 3 patients with grade 3 bilirubin increase) without any apparent dose-relationship. No grade 4 was reported.

Five patients had a grade 1 creatinine increased at various low doses, and no grade ≧2 was reported.

### 2.3. Determination of MTD and Dose Limiting Toxicities

A total of 34 patients have been treated in the dose escalation part of the study in 9 dose levels: 1 at 10 mg/m², 1 at 20 mg/m², 4 at 40 mg/m², 5 at 60 mg/m², 3 in each of the following DLs 90, 120 and 150 mg/m², 6 at 190 mg/m² and 8 at 240 mg/m².

All patients are evaluable for safety and dose limiting toxicity (DLT). DLT observation period was defined as the first cycle of study treatment. DLT as well as adverse events meeting the DLT criteria but observed after cycle 1 (subsequent cycles) are presented in Table 6.

TABLE 6
Toxicity defined as DLT (actual data) at cycle 1 and subsequent cycles

| SAR566658 | N patients | | Subsequent |
|---|---|---|---|
| dose level | treated | Cycle 1 | cycles |
| ≦60 mg/m² | 11 | — | — |
| 90 mg/m² | 3 | — | — |
| 120 mg/m² | 3 | — | — |
| 150 mg/m² | 3 | — | — |
| 190 mg/m² | 6 | — | — |
| 240 mg/m² | 8 | Diarrhea | Keratitis gr 3 at |
| | | Gr3 (037) | cy2 (033, 040) |

Gr: grade;
Cy: cycle

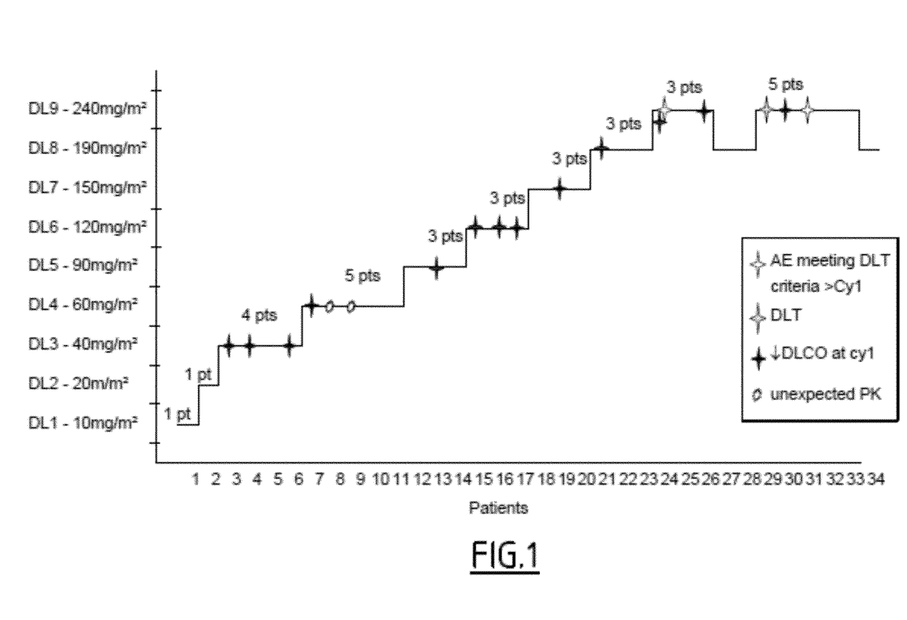

FIG. 1 summarizes patients treated by dose level and key events taken into account in the dose escalation determination.

As per protocol, an accelerated dose escalation scheme was used for the two first dose levels (DLs) and was based on toxicities observed during the first cycle of treatment. There were no related toxicities with grade ≧2 or changes in pulmonary function tests (PFTs) in patients treated at 10 mg/m² (DL1) and 20 mg/m² (DL2), allowing dose escalation to DL2 and then DL3. From this DL3 of 40 mg/m², the dose escalation proceeded with a classical scheme (3+3 design).

Three patients were treated at 40 mg/m². No DLT was observed. However, all patients experienced carbon monoxide diffusing capacity (DLCO) decrease at the end of cycle 1, three were confirmed at repeated tests 1 week later, but values were still within normal ranges. These decreases translated to grade 0 or 1 according to NCI-CTC 4.03. As stated in the protocol, external pneumologists were consulted. In the meantime, the study committee decided to allow inclusion of an additional patient at that DL. This patient had no DLT but also experienced a decrease in DLCO at the end of cycle 1. The expert review and assessment of these 4 patients was the following: “Decrease in DLCO >15% compared to baseline value is a significant change to evaluate lung toxicity. However, patient history is a major factor. In particular, advanced malignant disease and especially prior therapy known as potentially toxic for lung and received within 1 year before the tests (“recall” phenomenon) are confounding factors. In addition, the decrease has not significant value if still in the normal ranges”. These events of DLCO decreases were not considered as DLT by the experts but within expected range fluctuations and they recommended to pursue the dose escalation. This decision was further endorsed by the study committee.

Since the decision to proceed, three patients were treated at the fourth dose level (60 mg/m²). None of them experienced DLT. One patient (840002008) experienced a DLCO decrease >15% at the end of cycle 1 that occurred in a context of worsening of pleural effusion with passive atelectasis (disease progression). The 2 other patients did not experienced DLCO decrease >15%. However, 2 patients out of 3 had pharmacokinetics (PK) profile different from what was expected: the Cmax were as expected by dose proportionality but the AUC was lower. As a consequence it was decided to enrol 2 additional patients to obtain additional PK data at this DL. PK parameters (Cmax, AUC, CL and Vss) of these 2 last patients (4th and 5th) reflected what could have been expected for the 60 mg/m² dose level. Cmax, AUC, CL and Vss values reflect what could have been expected. The unexpected PK results of 2 patients (out of 5) at this same dose level remain without explanation so far and may results from inter-patients variability. No DLT and no DLCO decrease >15% were observed in the 2 additional patients treated at this DL4 (60 mg/m²). One patient experienced 2 severe AEs (SAEs) at cycle 1 in context of disease progression (non related grade 4 general health deterioration and grade 3 hyperbilirubinemia). This patient died from malignant disease on day 27 after having received his first infusion. Two patients at the DL4 (60 mg/m²) had non related grade 3 or 4 AEs observed in context of documented disease progression: one patient (described above) experienced portal vein thrombosis, hyperbilirubinemia, general health deterioration, and one patient had transaminases increase and alkalin phosphatase increase. Both patients had metastatic pancreatic cancer. The study committee agreed to escalate to the next dose level (DL5 90 mg/m²).

Three patients have been treated at each following dose level: 5th DL (90 mg/m²), 6th DL (120 mg/m²), 7th DL (150 mg/m²), and 8th DL (190 mg/m²). No DLT was reported, allowing the dose escalation at the subsequent DL. At 90 mg/m², one patient (840001015) experienced a DLCO decrease >15% at the end of cycle 1 that occurred in a context of worsening of pulmonary lymphangitis (disease progression). At 120 mg/m², one patient (724001022) experienced a DLCO decrease >15% at the end of cycle 1 that occurred in a context of worsening of patient's general condition, increase of ascites, and respiratory muscle weakness that could have explain this decrease in PFTs results, as per pneumologist report. At 150 mg/m² one patient (724001026) experienced a DLCO decrease >15% at the end of cycle 1 that was not confirmed at repeated test.

Three patients have been treated at the ninth dose level (240 mg/m²). None of them experienced DLT and no DLCO decrease >15% have been observed at the end of cycle 1. However, the first treated patient developed a grade 3 keratitis during cycle 2. This event occurred outside DLT observation period, but met DLT criteria and has been taken into account in the dose escalation determination process. Decision was taken to wait for cycle 2 completion for the 2nd and 3rd patients treated at 240 mg/m² in order to capture any severe ocular adverse event that could occur in cycle 2. In the meantime, 2 planned screened patients have been treated at the lowest DL 190 mg/m². They did not developed any DLT or DLCO decrease >15% at the end of cycle 1. Given the 2 last patients treated at 240 mg/m² DL have not developed any severe ocular toxicity during their second cycle of treatment, decision was taken to treat 3 more patients at 240 mg/m² DL. One of those additional patients experienced a DLT (Grade 3 diarrhea) at the end of cycle 1. Among the 6 patients treated at 240 mg/m², 1/6 patient experienced a DLT (Gr3 diarrhea) at cycle 1, 1/3 patient experienced Gr 3 keratitis at cycle 2 (at that time, one patient did not received cycle 1 and 2 patients had just received cycle 2 infusion). It was decided to follow a cautious approach by enrolling two more patients at the DL 240 mg/m² and follow safety until cycle 2 completion. In addition as a patient experienced nausea and vomiting at cycle 1 which did not met DLT criteria, prophylactic antiemetic drugs prior study treatment administration was recommended. Two additional patients were therefore treated at 240 mg/m². No DLT and no DLCO decrease >15% at the end of cycle 1 were observed. However, both 7^{th} and 8^{th} patients developed grade 2 keratitis. In addition the 6^{th} patient treated at this dose developed a grade 3 keratitis at cycle 2 leading to cycle 3 delay and dose decreased to 190 mg/m².

### CONCLUSION

At the highest dose of 240 mg/m², one patient out of the eight treated experienced a DLT (grade 3 diarrhea which recovered with symptomatic corrective treatment) at cycle 1. Among the 7 patients who received a second cycle, 2 experienced a grade 3 keratitis (which met DLT criteria), which lead to delay the administration of cycle 3 at a reduced dose. In addition, 4 other patients experienced a grade 2 keratitis at cycle 2 which led to cycle 3 delay in 3 patients. The DL 240 mg/m² was considered not feasible and was defined as the Maximum Administered Dose (MAD).

At DL 190 mg/m², five patients were treated; all received at least 2 cycles. Three patients developed a grade 2 keratitis: 2 patients at cycle 2 (including one patient who had a grade 2 keratitis before treatment administration which was clinically resolved on C1D1) and 1 patient at cycle 1 (knowing that patient reported dry eye from C1D1). The keratitis event leads to cycle 3 delay in one of those 3 patients. Even if 3 patients out of 5 treated at DL190 experienced a keratitis, this eye toxicity appeared to investigators less severe, more manageable with lower impact on study treatment compared to the one observed at DL240. In addition, 2 of those 3 patients had pre-existing eye abnormalities which could have impacted the ocular evaluation.

Therefore a 6^{th} patient was treated at 190 mg/m² to complete the enrolment at that dose. This patient did not develop any DLT.

The DL190 mg/m² was selected as the recommended dose.

### 2.4. Serious Adverse Events

Eight patients had at least one treatment emergent SAE, all considered not related to study treatment.

TABLE 7
Serious TEAE

| SAR566658 | | | |
|---|---|---|---|
| Dose level | Patient # | Cycle | SAE |
| 60 mg/m² | 008 | Cy 1 | Disease progression (NR) |
| 120 mg/m² | 022 | Cy 1 | Intestinal obstruction (NR) |
| 150 mg/m² | 023 | Cy 5 | Abdominal pain, back pain, intestinal |
| | | | obstruction (NR) |
| 190 mg/m² | 035 | Cy 2 | Metastases to CNS (NR) |
| | 036 | Cy 2 | Disease progression (NR) |
| | 029 | Cy 1 | Neck pain (NR) |
| | | Cy 2 | Abdominal pain, pulmonary |
| | | | embolism (NR) |
| 240 mg/m² | 038 | Cy 1 | Device related infection (NR) |
| | 033 | Cy 2 | Abdominal pain (NR) |
| | | Cy 4 | Abdominal pain, GI hemorrhage (NR) |

NR: not related to study treatment;
Cy: cycle;
CNS central nervous system;
GI gastrointestinal

### 2.5. Deaths

Of the 34 treated patients, 9 patients have a death documented. According to investigators, all patients died from malignant disease. Two patients (#008 and 036) died within 30 days from the last infusion, on cycle 1 day 27 and cycle 2 day 18, respectively.

### 2.6. Other Safety Measures: Specific Safety

### 2.6.1. Ocular Toxicity

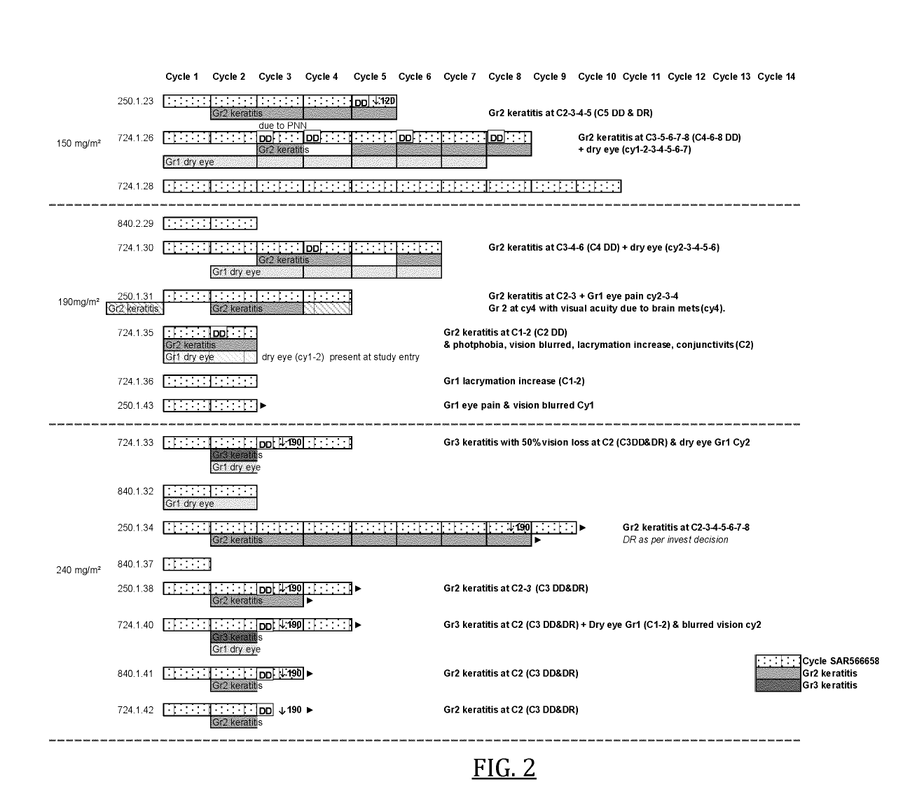

Ocular adverse events were mainly reported from 150 mg/m², as observed with other maytansinoid-loaded ADCs (Table 8 and FIG. 2).

Related ocular events included: dry eye, blurred vision, keratitis, photophobia, lacrimation increase, eye pain. Overall 15 patients (44.1%) had at least one related ocular event with the following severity: grade 1 in 3 patients, grade 2 in 10 patients, and grade 3 in 2 patients. Few other mild to moderate ocular events considered not related to study treatment were reported: ocular rosacea, eye discharge, and viral conjunctivitis.

Bilateral keratitis was one of the main ocular event observed with SAR566658. This event was often preceded by symptoms such as mild to moderate dry eye, blurred vision or photophobia. Those preliminary symptoms were mainly observed at cycle 1, whereas the diagnosis of keratitis was given later during the second or subsequent cycles of study treatment. The ophthalmological report usually described a superficial keratitis with corneal depots saving the central corneal zone. An epithelial inflammation (or stromal inflammation) has been reported only in the 2 severe cases at 240 mg/m². Topical treatment was started and included artificial tears and corticosteroid. So far recovery of the symptoms was observed within 1 to 3 weeks depending on the initial severity. If symptoms were still present on day 21 of a given cycle, the following cycle was delayed and as soon as symptoms disappeared, and provided that the lesions observed with the slip lamp were stable, the ophthalmologist gave green light to resume the treatment.

As no grade 1 keratitis exists in the NCI CTC v4.03, all the keratitis were graded 2. However among this category of grade 2, there are superficial keratitis with associated symptoms and without symptoms. Even if the keratitis appeared ongoing throughout several cycles with the same grade 2, the keratitis improved enough to allow administration of study treatment but it does not reflect in a grade change. Indeed, this classification does not allow to capture improvement of grade 2.

Two patients experienced a Gr 3 ocular toxicity during cycle 2 at 240 mg/m². The event started between day 8 and day 15 of the 2^{nd} cycle with loss of visual acuity, blurred vision and dry eyes. The ophthalmologist documented a Gr 3 bilateral keratitis with linear depot saving the central zone of cornea. Artificial drops and topical corticosteroids were given. At the end of cycle 2 visit (day 21) a partial recovery of vision loss and symptoms were noted and the ophthalmologist reported an improvement of keratitis (from Gr 3 to Gr 2). Cycle 3 was delayed in both patients by 2 weeks, and was administered at reduced dose (190 mg/m²).

Differences were observed across dose levels, in term of incidence, grade or impact on study treatment. Highest incidence, worst grade and highest impact on study treatment were observed at the highest dose level tested (240 mg/m²). No difference was observed in term of cycle of occurrence.

So far, all related ocular AEs recovered or were rapidly manageable allowing continuation of treatment with local treatment (artificial tears, and corticosteroid).

TABLE 8
Worst grade related ocular AEs during treatment (per patient and per cycle)

| | | | | | | Cy |
|---|---|---|---|---|---|---|
| SAR566658 | N pts with | | | | | delay |
| dose level | ocular tox | Patient # | Cycle | Ocular AE | Outcome | Dose ↓ |
| ≦60 mg/m² | 2 pts/11 | 004 | Cy1 | Vision blurred | recovered | |
| | | | | Gr1 | | |
| | | 007 | Cy1 | Dry eye Gr2 | recovered | |
| 120 mg/m² | 0/3 | — | | | | |
| 150 mg/m² | 2 pts/3 | 023 | Cy2-3-4-5 | Keratitis Gr2 | recovered | DD-DR |
| | | 026 | Cy1-2-3-4-5- | Dry eye Gr1 | recovered | DDx3 |
| | | | 6-7 | | | |
| | | | Cy3-5-6-7-8 | Keratitis Gr2 | recovered | |
| 190 mg/m² | 5 pts/6 | 031 | Cy2-3 | Keratitis Gr2 | recovered | |
| | | 030 | Cy2-3-4-5-6 | Dry eye Gr1 | recovered | |
| | | | Cy3-4-6 | Keratitis Gr2 | recovered | DD |
| | | 035 | Cy1-2 | Keratitis Gr2 | recovered | DD |
| | | | Cy2 | Lacrymation | recovered | |
| | | | | increase Gr1 | | |
| | | | | Photophobia Gr1 | recovered | |
| | | 036^{a} | Cy1-2 | Lacrymation | Not | |
| | | | | increase Gr1 | recovered | |
| | | 043^{b} | Cy1 | Eye pain Gr1 | recovered | |
| | | | Cy2 | Vision blurred | Not | |
| | | | | Gr1 | recovered | |
| 240 mg/m² | 6 pts/8 | 034^{b} | Cy2-3-4-5-6- | Keratitis Gr2 | Not | |
| | | | 7-8 | | recovered | |
| | | 038^{b} | Cy2-3 | Keratitis Gr2 | Not | DD-DR |
| | | | | | recovered | |
| | | 033 | Cy2 | Dry eye Gr2 | recovered | |
| | | | | Keratitis Gr3 | recovered | DD-DR |
| | | | | Vision blurred | recovered | |
| | | | | Gr2 | | |
| | | 040 | Cy1-2 | Dry eye Gr1 | recovered | |
| | | | Cy2 | Keratitis Gr3 | recovered | DD-DR |
| | | | | Vision blurred | recovered | |
| | | | | Gr3 | | |
| | | 041 | Cy2 | Keratitis Gr2 | recovering | DD-DR |
| | | 042 | Cy2 | Keratitis Gr2 | recovered | DD-DR |

| | | |
|---|---|---|
| Total | 15 pts/34 | Median cycle of occurrence keratitis: |
| | | keratitis (11 pts) cy2 [1-3]; dry eye (6 pts) cy 1 [1-2] |

^{a}event ongoing at time of death within 30 day from last IP
^{b}still under treatment

### 2.6.2. Peripheral Neuropathy

Twelve (35%) patients had either peripheral neuropathy (HLT) or paresthesia/dysesthesia (HLT) during study treatment. All were mild to moderate in intensity, and none of those events led to study treatment delay or discontinuation. Of note all patients were previously pre-treated with chemotherapy including one or a combination of the following compounds: oxaliplatin, cisplatin, carboplatin, paclitaxel or docetaxel. In addition, 2 patients had peripheral neuropathy at study entry (021 and 035).

The neurological event was attributed to study treatment in 8 patients.

No clear dose-dependency was observed regarding peripheral neuropathy (including paresthesia and dysesthesia).

TABLE 9
Worst grade peripheral neuropathy by initial planned dose
(per pt and per cy)

| | N of | | | | | Any periph | |
|---|---|---|---|---|---|---|---|
| SAR566658 | evalu- | Peripheral | | Paresthesia/ | | neuro event | |
| Dose | able | neuropathy^{a} | | dysaesthesia^{b} | | All | |
| level | patients | All Gr | Gr3-4 | All Gr | Gr3-4 | Gr | Gr3-4 |
| ≦60 mg/m² | 11 | 2 | 0 | 0 | 0 | 2 | 0 |
| 90 mg/m² | 3 | 0 | 0 | 1 | 0 | 1 | 0 |
| 120 mg/m² | 3 | 1 | 0 | 0 | 0 | 1 | 0 |
| 150 mg/m² | 3 | 2 | 0 | 0 | 0 | 2 | 0 |
| 190 mg/m² | 6 | 1 | 0 | 0 | 0 | 1 | 0 |
| 240 mg/m² | 8 | 4 | 0 | 2 | 0 | 5 | 0 |
| Total | 34 | 10 | 0 | 3 | 0 | 13 | 0 |
| | | (29.4%) | | | | (35.3%) | |

^{a}HLT peripheral neuropathies NEC (neuropathy peripheral, peripheral sensory neuropathy).
^{b}HLT

### 2.6.3. Lung Toxicity

As per protocol, pulmonary function tests were performed at study entry and at the end of each cycle. At the end of cycle 1 (end of DLT observation period), PFTs results were sent to external pneumologist to get advice about potential lung toxicity.

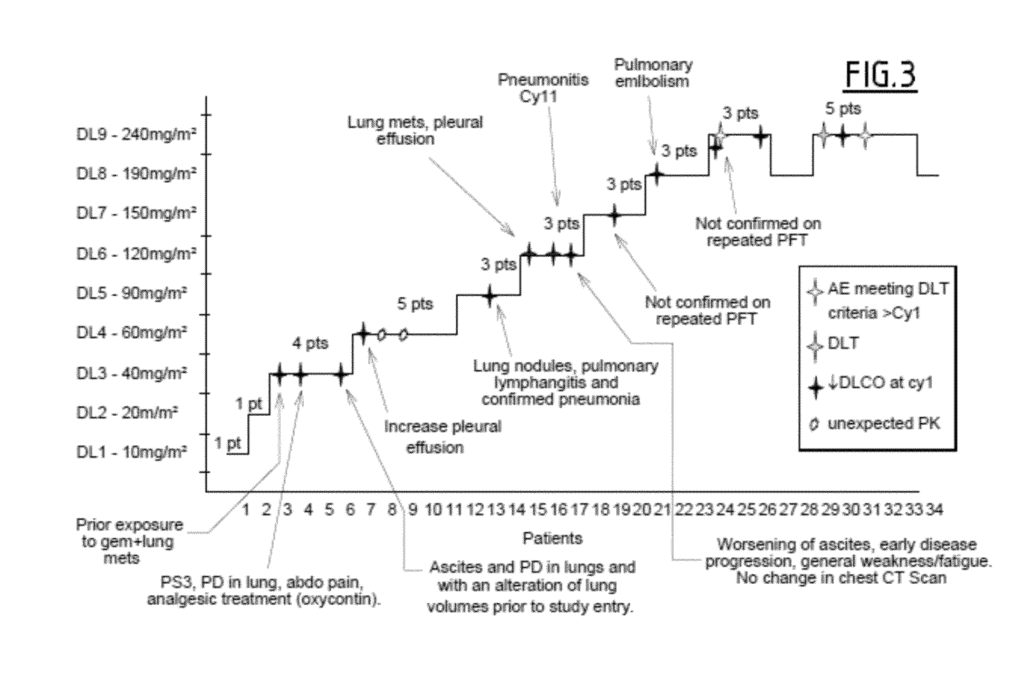

So far no PFTs abnormalities observed at cycle 1 has been attributed to lung toxicity (FIG. 3).

One interstitial pneumonitis has been observed in a patient treated at 120 mg/m². This patient (724001021) with an metastatic ovarian cancer (pelvic lymph nodes and peritoneal involvement) received a total of 14 cycles and developed pulmonary symptoms with grade 1 dyspnea and cough at cycle 12. Chest CT Scan performed during the same cycle showed lung abnormalities. In addition PFTs tests showed a decrease in DLCO by approximately 14%. Therefore study treatment was delayed and steroids and antibiotics were prescribed. Patient felt better with less dyspnea and cough. A new chest CT scan confirmed the previous radiological findings and the lung lesions were described by the radiologist not clearly disease related but possible relation to study treatment could not be excluded. PFTs showed again a DLCO decrease of about 16% in comparison to baseline. Decision to perform a broncoscopy with broncoalveolar lavage was taken. The antibiotics and steroids were continued.

On March 12, after 2 weeks of treatment delay, patient felt better, cough and dyspnea improved. Microbiological examination following the broncho alveolar lavage was negative, no tumoral cell was found and cytological exam showed neutrophil and eosinophil infiltration. Due to these findings study treatment relationship could not be ruled out. However, in consideration of the good general condition, the improvement of respiratory symptoms and the benefit achieved on her tumor, Investigator's decision in agreement with sponsor was to continue with treatment at the same dose (as per protocol), despite the decrease of DLCO within 10-20% in comparison with the baseline. Cycle 13 and cycle 14 were administered. Cough and dyspnea recovered during cycle 14. CT Scan showed disease progression (increase of lymph node lesions) and treatment was discontinued. DLCO decrease in comparison with the baseline was approximately of 35%. He denied respiratory symptoms. Interstitial pneumonia was considered resolved by the investigator 45 days after the last infusion.

### 3. Efficacy Results

Antitumoral clinical activity has been observed from doses ≧120 mg/m², i.e. tumor sizes decrease for radiologically assessable lesions or long stabilisation or improvement of tumor related symptoms (such as pain . . . ).

Among the 33 patients evaluable for tumor response, one confirmed PR, and 15 stable diseases are reported (Table 10). SD and PR were mainly observed at doses ≧120 mg/m². Indeed among the 19 patients evaluable for response at those doses, 13 SD (including 2 unconfirmed PR and 1 PR to be confirmed) and 1 PR were observed.

Of note, those PR/SD by tumor type whatever the dose are as follows:
2 SD of short duration in 9 pancreas,
1PR and 1 unconfirmed PR (i.e. SD) in 4 breasts (knowing that the 2 non responsive patients were treated at 10 and 40 mg/m² respectively),
7 SD (including one unconfirmed PR and 1 PR to be confirmed) in 13 evaluable ovarian cancers.

The PR was reported in a 63-year-old breast cancer patient (724001026) treated at 150 mg/m². The PR was observed at cycle 2, confirmed at cycle 4 and 6 with a maximum decrease in target lesions of 57%. She had at study entry 2 liver target lesions and multiples lung non target lesions. Prior anticancer therapy included 3 prior lines of chemotherapies: pegylated doxorubicin-cyclophosphamide, then paclitaxel and an investigational drug (IND) for 5 months, then gemcitabine and an IND for 1 month. This breast tumor is a triple negative, not positive to receptors for estrogen, progesterone, or HER2. CA6 expression as per IHC on archival tumor (1 year before study entry) showed 70% 3+ membrane staining. She received a total of 8 cycles of study treatment and discontinued due to documented liver disease progression (increase of target lesions and occurrence of new lesions).

Among the 15 SDs, the investigators reported 2 unconfirmed PRs and 1 PR to be confirmed:
One patient (#724001021) at 120 mg/m²: ovarian cancer, 65-year-old, pretreated with 3 prior lines of chemotherapy (paclitaxel-carboplatin for 7 months, topotecan for 4 months and pegylated doxorubicin for 1 month). She had at study entry peritoneum and lymph nodes involvement. A regular decrease of target lesions was observed during study treatment with maximum observed at cycle 10: 28.8% at Cycle 8, 36.6% at Cycle 10 and 27.6% at Cycle 12, compared to baseline evaluation. Disease progression on target lesions was observed at cycle 14 and patient discontinued from study treatment. CA6 expression as per IHC on archival tumor (12 years before study entry) showed 40% 3+ membrane staining
One patient (#250001031) at 190 mg/m²: breast cancer, 49-year-old, pretreated with several prior anticancer treatments (fluorouracile-epirubicin-cyclophosphamide, capecitabine, methotrexate-endoxan, docetaxel, navelbine, eribulin as well as hormone therapy). She had at study entry liver, lymph nodes and bone involvement. A decrease of target lesions was observed at cycle 2 (55%) still present at cycle 4 (71%) but carcinomatous meningitis was diagnosed at cycle 4 and treatment was discontinued. CA6 expression as per IHC on archival tumor (6 years before study entry) showed 50% 2+ membrane staining
One patient (840001041) at 240 mg/m²: ovarian cancer, 67-year-old, diagnosed in October 2010, then treated with surgery and adjuvant chemotherapy (paclitaxel-carboplatin). Lymph node relapse was diagnosed in January 2013 and she entered the trial. A decrease of target lesions was observed at cycle 2 (35%) and should be confirmed at cycle 4. In addition a decrease of tumor marker from 39.5 to 11.3 UI/L (CA125) was reported. CA6 expression as per IHC showed 15% 2+ and 35% 3+ membrane staining.

In addition, investigator reported an improvement of general status observed from cycle 1 in one 58-year-old male patient with bladder cancer. At study entry he had pelvic lymph node involvement responsible for bilateral limb edema. CT Scan showed stabilization up to cycle 10 where new lesions were observed and patient discontinued from study treatment.

TABLE 10
Best overall response

| SAR566658 | N patients | | | | |
|---|---|---|---|---|---|
| Dose level | treated | PR | SD | PD | NE |
| ≦60 mg/m² | 11 | 0 | 1 | 10 | — |
| 90 mg/m² | 3 | 0 | 1 | 2 | — |
| 120 mg/m² | 3 | 0 | 2 | 1 | — |
| | | | #021 PR not | | |
| | | | conf | | |
| 150 mg/m² | 3 | 1 | 2 | 0 | — |
| | | #026 Breast | | | |
| | | cancer | | | |
| 190 mg/m² | 5 | 0 | 2 | 3 | 1* |
| | | | #031 PR not | | (#043) |
| | | | conf | | |
| 240 mg/m² | 8 | 0 | 7 | 1 | 0 |
| Total | 33 | 1 | 15 | 17 | 1* |

*too early, patients not yet evaluable

### 4. Pharmacokinetic (PK) Results

Parallel elimination profile of SAR566658 with t_{1/2z} around 5 Days
Exposure to SAR566658 (Cₘₐₓ and AUC) increased with no major deviation from dose proportionality over the dose range 10 to 240 mg/m²
Clearance was roughly constant over the dose range 20 to 240 mg/m² ranging between 0.5 and 0.9 L/day except for 2 patients treated at 60 mg/m² (CL˜1.5-2 L/day)
Overall, total variability is low to moderate

1: A method for treating cancer in a patient comprising administering to the patient a conjugate comprising (i) a cell binding agent which binds to the extracellular domain of the human mucin-1 (MUC1) glycoprotein, linked to (ii) at least one cytotoxic agent, wherein the conjugate is administered to the patient at a dose of at least 120 mg/m².

2. (canceled)

3: The method of claim 1, wherein the cell binding agent recognizes and binds the CA6 glycotope on the MUC1 glycoprotein.

4: The method of claim 1, wherein the cell binding agent is an antibody or an epitope-binding fragment thereof.

5: The method of claim 4, wherein the antibody or epitope-binding fragment thereof comprises one or more complementarity-determining region (CDR) having an amino acid sequence selected from the group consisting of SEQ ID NO: 1, SEQ ID NO: 2, SEQ ID NO: 3, SEQ ID NO: 4, SEQ ID NO: 5, and SEQ ID NO: 6.

6: The method of claim 5, wherein the antibody or epitope-binding fragment thereof comprises a CDR1-H of amino acid sequence SEQ ID NO: 1, a CDR2-H of amino acid sequence SEQ ID NO: 2, a CDR3-H of amino acid sequence SEQ ID NO: 3, a CDR1-L of amino acid sequence SEQ ID NO: 4, a CDR2-L of amino acid sequence SEQ ID NO: 5, and a CDR3-L of amino acid sequence SEQ ID NO: 6.

7: The method of claim 4, wherein the antibody or epitope-binding fragment thereof comprises a heavy chain variable region having the amino acid sequence of SEQ ID NO: 7 or an amino acid sequence that is at least 85% identical to the amino acid sequence of SEQ ID NO: 7.

8: The method of claim 4, wherein the antibody or epitope-binding fragment thereof comprises a light chain variable region having the amino acid sequence of SEQ ID NO: 8 or an amino acid sequence that is at least 85% identical to the amino acid sequence of SEQ ID NO: 8.

9. (canceled)

10: The method of claim 1, wherein the cell binding agent is a monoclonal antibody comprising a heavy chain having the amino acid sequence of SEQ ID NO: 9 or an amino acid sequence that is at least 85% identical to the amino acid sequence of SEQ ID NO: 9, and a light chain having the amino acid sequence of SEQ ID NO: 10 or an amino acid sequence that is at least 85% identical to the amino acid sequence of SEQ ID NO: 10.

11: The method of claim 1, wherein the at least one cytotoxic agent is the maytansine DM4 of formula (II)

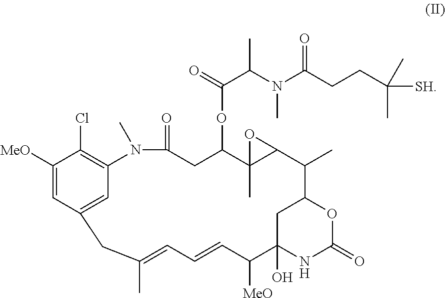 [SMILES: COc1cc2cc(c1Cl)N(C)C(=O)CC(OC(=O)C(C)N(C)C(=O)CCC(C)(C)S)C1(C)OC1C(C)C1CC(O)(NC(=O)O1)C(OC)/C=C/C=C(\C)C2]

12-13. (canceled)

14: The method of claim 1, wherein the cell binding agent is covalently linked via a cleavable or non-cleavable linker to the at least one cytotoxic agent, and wherein the linker is N-succinimidyl pyridyldithiobutyrate (SPDB) and the cytotoxic agent is DM4.

15-17. (canceled)

18: The method of claim 1, wherein the conjugate is characterized by a drug-to-antibody ratio (DAR) ranging from 3 to 4, the DAR being calculated from the ratio of the cytotoxic agent concentration (C_{D}) to that of the cell binding agent (C_{A});
$$DAR = \frac{{c}_{D}}{{c}_{A}}$$
$$wherein$$
$${C}_{D} = [ ( {ɛ}_{A 280} \times {A}_{252} ) - ( {ɛ}_{A 252} \times {A}_{280} ) ] / [ ( {ɛ}_{D 252} \times {ɛ}_{A 280} ) - ( {ɛ}_{A 252} \times {ɛ}_{D 280} ) ]$$
$${C}_{A} = [ {A}_{280} - ( {C}_{D} \times {ɛ}_{D 280} ) ] / {ɛ}_{A 280}$$
and
ε_{D252} and ε_{D280} are respectively the molar extinction coefficients of the cytotoxic agent at 252 nm and 280 nm,
ε_{A252} and ε_{A280} are respectively the molar extinction coefficients of the cell binding agent at 252 nm and 280 nm, and
A₂₅₂ and A₂₈₀ are respectively the absorbances for the conjugate at 252 nm (A₂₅₂) and at 280 nm (A₂₈₀), measured using a classic spectrophotometer apparatus.

19: The method of claim 1, wherein the conjugate is administered at a dose ranging from 150 mg/m² to 240 mg/m².

20: The method of claim 19, wherein the conjugate is administered at a dose of 190 mg/m².

21. (canceled)

22: The method of claim 1, wherein the conjugate is administered to the patient in an intermittent program with an interval between each administration, and wherein the median number of cycles of administration is 2.

23: The method of claim 1, wherein the conjugate is administered intravenously.

24. (canceled)

25: The method of claim 1, wherein the cancer is a CA6-positive solid tumor.

26. (canceled)

27: The method of claim 25, wherein the cancer is selected from the group consisting of breast cancer and ovarian cancer.

28. (canceled)

29: The method of claim 27, wherein the breast cancer is a triple negative breast cancer that is not positive to receptors for estrogen, progesterone, or HER2.

30: The method of claim 1, wherein the patient treated has been previously treated with an oxaliplatin-, cisplatin-, a carboplatin-, and/or a paclitaxel-, docetaxel-based regimen.

31: The method of claim 1, wherein the patient is also treated with either a keratitis prophylactic or curative ocular composition.

32: A conjugate comprising (i) a cell binding agent which binds to the extracellular domain of the human mucin-1 (MUC1) glycoprotein, linked to (ii) at least one cytotoxic agent, wherein the conjugate is effective for treating either breast cancer or ovarian cancer.

33. (canceled)

34: An article of manufacture comprising:
a) a packaging material;
b) a conjugate comprising (i) a cell binding agent which binds to the extracellular domain of the human mucin-1 (MUC1) glycoprotein, linked to (ii) at least one cytotoxic agent; and
c) a label or package insert contained within the packaging material indicating that the conjugate is administered at a dose of at least 120 mg/m².

35: An article of manufacture comprising:
a) a packaging material;
b) a conjugate comprising (i) a cell binding agent which binds to the extracellular domain of the human mucin-1 (MUC1) glycoprotein, linked to (ii) at least one cytotoxic agent; and
c) a label or package insert contained within the packaging material indicating that the conjugate is administered for treating either breast cancer or ovarian cancer.
